(19) United States
(12) Patent Application Publication
(10) Pub. No.: US 2009/0197279 A1
(43) Pub. Date: Aug. 6, 2009

(54) MICROTUBULE SYNTHESIS AS A BIOMARKER

(75) Inventors: Mark K. Hellerstein, Kensington, CA (US); Patrizia A. Fanara, Oakland, CA (US)

Correspondence Address: MORGAN, LEWIS & BOCKIUS, LLP, ONE MARKET SPEAR STREET TOWER, SAN FRANCISCO, CA, 94105, US

(73) Assignee: KINEMED, INC., Emeryville, CA (US)

(21) Appl. No.: 12/088,663

(22) PCT Filed: Oct. 2, 2006
(86) PCT No.: PCT/US06/38710

§ 371 Date: Jan. 8, 2009

Related U.S. Application Data
(60) Provisional application No. 60/722,897, filed on Sep. 30, 2005.

(51) Int. Cl. G01N 33/53; C12Q 1/02

(52) U.S. Cl. 435/7.1; 435/29

(57) ABSTRACT

Stable isotope labeling was used to measure dynamics of tubulin incorporation into microtubule subpopulations representing different neuronal compartments in the murine hippocampus. Neuronal microtubules were largely static. Basal turnover was highest in tau-associated (axona) and growth cone), lower in MAP2-associated (somatodendritic), and lowest in cold stable (axonal shaft) subpopulations. Intracerebroventricular glutamate injection stimulated label incorporation into axonal shaft and somatodendritic microtubules, the latter dependent on cAMP-PKA. Hippocampus-dependent memory formation after contextual fear conditioning was accompanied by increased assembly of MAP2- and cold stable-microtubules. Both microtubule assembly and memory formation were inhibited by the microtubule depolymerizing drug, nocodazole. This approach allows for correlation with behavioral measures of learning and memory and for the screening of candidate agents for stimulatory activities on learning memory.

### CROSS-REFERENCES TO RELATED APPLICATIONS

This application claims priority to U.S. provisional application No. 60/722,897 filed on Sep. 30, 2005 which is hereby incorporated by reference in its entirety.

### FIELD OF THE INVENTION

The invention relates to methods for measuring changes in biochemical processes that may underlie memory, learning, and other neurobiological changes in the brain including various diseases and disorders such as Alzheimer's disease. More specifically, the invention relates to measuring the turnover of microtubule polymers within neurons and other cell types comprising nerve tissue.

### BACKGROUND OF THE INVENTION

The neuronal basis of learning involves the creation of synaptic connections (i.e., synaptogenesis) and changes in synaptic strength (i.e. rapid protein synthesis). Structural plasticity or morphologic changes in synapses are particularly important in long-term potentiation (i.e., synaptic plasticity) and are fundamental to the learning and memory process. Dendritic spines and axonal connections appear to be dynamic structures that underlie learning and memory formation. The biochemical basis of synaptic connections and remodeling is the formation of microtubules (polymers of tubulin). These connections are maintained by the remarkable stability of dendritic and axonal microtubules.

Thus, learning and memory may have a specific biochemical correlate, namely the synthesis and stability of new microtubules in dendrites and axons. Loss of memory traces similarly can be represented in biochemical terms, as the breakdown of pre-formed polymers of tubulin. The notion that memory traces have a measurable biochemical correlate (turnover of polymers of tubulin) suggests a number of enormously promising applications in neurobiology and neurologic disease, if this biochemical process could be measured in vivo.

### SUMMARY OF THE INVENTION

The invention is directed toward measuring microtubule polymer stability in cells and tissues of the brain by measuring the molecular flux rates of microtubule synthesis and degradation (microtubule dynamics). Tubulin is labeled with an isotope and dimers and polymers of tubulin are isolated and measured for isotopic enrichment using mass spectrometry or other appropriate techniques, e.g., liquid scintillation counting if the isotope is radioactive.

The invention allows for the comparison between the rates of polymerization and depolymerization of microtubules (i.e., microtubule dynamics) measured from neurons, central nervous system (CNS) tissues, or organisms that have been exposed to one or more compounds (or combinations or mixtures thereof) to the rates of polymerization and depolymerization of microtubules measured from non-exposed neurons, CNS tissues, or organisms. Non-exposed neurons, CNS tissues, or organisms may be neurons, CNS tissues, or organisms having a disease such as dementia or a learning disorder but not yet having been exposed to one or more compounds (or combinations or mixtures thereof), or non-exposed neurons, CNS tissues, or organisms may be neurons, CNS tissues, or organisms not having dementia or a learning disorder. Differences between the rates of polymerization and depolymerization of microtubules from the exposed and non-exposed neurons, CNS tissues, or organisms are identified and this information is then used to determine whether the one or more compounds (or combinations or mixtures thereof) elicit a change in microtubule dynamics in the exposed neuron, CNS tissue, or organism. The one or more compounds (or combinations or mixtures thereof) may be administered to a mammal and the microtubule dynamics (rates) calculated and evaluated against the dynamics (rates) calculated from an unexposed mammal of the same species. The neurons may be cultured or may be isolated from an organism. The CNS tissue may be ex vivo or isolated from an organism after the organism has been exposed to the one or more compounds (or combinations or mixtures thereof). The mammal may be a human.

In one embodiment, microtubule dynamics are measured by use of stable isotope labeling techniques. Said techniques involve the administration or contacting of stable isotope-labeled substrates to a biological system of interest.

The stable isotope label may include ²H, ¹³C, ¹⁵N, ¹⁸O, ³³S, ³⁴S. In another embodiment, the microtubule dynamics (rates) are measured by use of radioactive isotope labeling techniques. The radioactive isotope may include ³H, ¹⁴C, ³²P, ³³P, ³⁵S, ¹²⁵I, ¹³¹I.

Isotope-labeled substrates include, but are not limited to, ²H₂0, H₂¹⁸O, ¹⁵NH₃, ¹³CO₂, H¹³C0₃, ²H-labeled amino acids, ¹³C-labeled amino acids, ¹⁵N-labeled amino acids, ¹⁸O-labeled amino acids, ³⁴S or ³³S-labeled amino acids, ³H₂O, ³H-labeled amino acids, and ¹⁴C-labeled amino acids, ²H-glucose, ¹³C-labeled glucose, ²H-labeled organic molecules, ¹³C-labeled organic molecules, and ¹⁵N-labeled organic molecules.

In one embodiment, the incorporation of stable isotope-labeled substrates into one or more tubulin dimers (subunits of microtubule polymers) and the incorporation into microtubule polymers are measured concurrently by methods known in the art. In this manner, the dynamics of microtubules can be determined by measuring and comparing, over specific time intervals, the isotopic content and/or pattern or the rate of change of the isotopic content and/or pattern in the tubulin dimer or microtubule, for example by using mass spectrometry or other analytical techniques known in the art. The relationship between the isotopic content and/or pattern or the rate of change of the isotopic content and/or pattern in the microtubule to the isotopic content and/or pattern or rate of change in the isotopic content and/or pattern in the unassembled tubulin dimer subunits may be particularly informative. The dynamics of microtubule assembly and disassembly (polymerization and depolymerization) can then be calculated.

Alternatively, radiolabeled substrates are contemplated for use in the present invention wherein the radiolabeled substrates are incorporated into tubulin dimers, which are then incorporated into microtubule polymers. In this manner, the dynamics of microtubules can be determined by measuring radioactivity present in the tubulin dimers and microtubule polymers by using techniques known in the art such as scintillation counting. The dynamics of microtubule polymers are then calculated, using methods known in the art.

In another embodiment, the dynamics of microtubules are measured from the assembly and disassembly (polymerization and depolymerization) of microtubules in a living organism prior to, and after, exposure to one or more compounds, to evaluate toxicity, e.g., memory or learning impairment. In one variation, the one or more compounds may be industrial or occupational chemicals. In another variation, the one or more compounds may be cosmetics. In yet another variation, the one or more compounds may be food additives. And in yet another variation, the one or more compounds may be environmental pollutants.

Alternatively, exposure of one or more compounds may be to one living organism and the dynamics of microtubules are compared to the dynamics of microtubules from an unexposed living organism of the same species to evaluate toxicity.

The measurement of microtubule dynamics in differentiated neurons, which does not reflect proliferation rates since neurons are post-mitotic, allows for the measurement of important biological processes such as axonal dysfunction, which could affect learning and memory, as is more fully described, infra.

In another embodiment of the invention, isotopically-perturbed molecules are provided, said isotopically-perturbed molecules comprising one or more stable isotopes. The isotopically-perturbed molecules are products of the labeling methods described herein.

In yet another embodiment of the invention, the isotopically-perturbed molecules are labeled with one or more radioactive isotopes.

In yet another embodiment of the invention, one or more kits are provided that comprise isotope-labeled precursors and instructions for using them. The kits may contain stable-isotope labeled precursors or radioactive-labeled isotope precursors or both. Stable-isotope labeled precursors and radioactive-labeled isotope precursors may be provided in one kit or they may be separated and provided in two or more kits. The kits may further comprise one or more tools for administering the isotope-labeled precursors. The kits also may comprise one or more tools for collecting samples from a subject.

In yet another embodiment of the invention, one or more information storage devices are provided that comprise data generated from the methods of the present invention. The data may be analyzed, partially analyzed, or unanalyzed. The data may be imprinted onto paper, plastic, magnetic, optical, or other medium for storage and display.

In yet another embodiment of the invention, one or more drug candidates identified and at least partially characterized by the methods of the present invention are contemplated.

In one embodiment, the invention is comprised of the following steps: (1) administer an isotope label in vivo to an animal wherein said label is incorporated into one or more tubulin dimers; (2) collect brain tissue from the animal; (3) isolate one or more isotope-labeled tubulin dimers and/or one or more isotope-labeled tubulin polymers from the brain tissue; (4) determine isotopic enrichment of the one or more tubulin dimers and/or one or more tubulin polymers or their amino acid components; (5) measure the content, rate of incorporation and/or pattern or rate of change in content and/or pattern of isotope labeling of said one or more isotope-labeled tubulin dimers incorporated into said one or more tubulin polymers; (6) measure the content, rate of incorporation and/or pattern or rate of change in content and/or pattern of isotope labeling of one or more free tubulin dimers; (7) calculate molecular flux rates in the incorporation of one or more tubulin subunits incorporated into one or more tubulin polymers based on the content and/or pattern or rate of change of content and/or pattern of isotopic labeling in said one or more tubulin dimers incorporated into said one or more tubulin polymers; and (8) compare the molecular flux rates between said one or more tubulin dimers incorporated into said one or more tubulin polymers with the molecular flux rates between said one or more free tubulin dimers. The isotope label may be labeled water. In one embodiment, the labeled water is ²H₂O. In another embodiment, the labeled water is H₂¹⁸O. In yet another embodiment, the labeled water is ³H₂O. In another embodiment, the isotope label may include specific heavy isotopes of elements present in biomolecules, such as ²H, ¹³C, ¹⁵N, ¹⁸O, ³³S, ³⁴S, or may contain other isotopes of elements present in biomolecules such as ³H, ¹⁴C, ³⁵S, ¹²⁵I, ¹³¹I. The isotope label may be isotope-labeled protein precursors including, but not limited to, ²H₂0, ¹⁵NH₃, ¹³CO₂, and H¹³C0₃. The isotope label may be labeled amino acids including, but not limited to, ²H-labeled amino acids, ¹³C-labeled amino acids, ¹⁵N-labeled amino acids, ¹⁸O-labeled amino acids, ³⁴S or ³³S-labeled amino acids, ³H₂O ³H-labeled amino acids, and ¹⁴C-labeled amino acids. In one embodiment, the labeled amino acid is ²H₃-leucine. In another embodiment, the labeled amino acid is ¹⁵N-glycine.

The ²H label enters newly synthesized free tubulin subunits via metabolic pathways for the biosynthesis of nonessential amino acids. Incorporation of ²H into newly synthesized free tubulin dimers appears in the tubulin dimer pool before being incorporated through polymerization into microtubules (see FIG. 2). In biological settings in which microtubules are highly dynamic, ²H-label accumulates in the dimer and polymer pools at similar rates reflecting rapid exchange kinetics between the two pools (i.e., dynamic instability of the microtubules). However, in settings where microtubules are stabilized by MAPs (e.g., the microtubules within neurons), ²H-label in newly synthesized tubulin appears in the dimer pool at a rate proportional to the biosynthetic rate of free tubulin, whereas incorporation into microtubule polymers is slower, often dramatically so.

Microtubule dynamics are then quantified by gas chromatography/mass spectrometry (GC/MS) or other analytical techniques known in the art and discussed more fully, infra.

The present invention provides methods for evaluating the effect on synaptic plasticity or synaptogenesis. In one embodiment, the method includes a) exposing a living system to one or more candidate agents; b) administering an isotope-labeled substrate to the living system for a period of time sufficient for the isotope-labeled substrate to enter into one or more tubulin subunits in neurons and thereby enter into and label one or more populations of microtubule molecules; c) obtaining one or more samples from the living system, wherein said one or more samples comprises at least one isotope-labeled subunit incorporated into said one or more populations of microtubule molecules; d) obtaining one or more samples from the living system, wherein the sample(s) have at least one isotope-labeled free tubulin subunit; e) measuring the content, rate of incorporation and/or pattern or rate of change in content and/or pattern of isotope labeling of the isotope-labeled tubulin subunit(s) incorporated into the population(s) of microtubule molecules; f) measuring the content, rate of incorporation and/or pattern or rate of change in content and/or pattern of isotope labeling of the isotope-labeled free tubulin subunit(s); g) calculating molecular flux rates in the incorporation of the isotope-labeled tubulin subunit(s) incorporated into the population(s) of microtubule molecules based on the content and/or pattern or rate of change of content and/or pattern of isotopic labeling in the isotope-labeled tubulin subunit(s) incorporated into the population(s) of microtubule molecules; h) calculating molecular flux rates in the incorporation of the isotope-labeled free tubulin subunit(s) of the population(s) of microtubule molecules based on the content and/or pattern or rate of change of content and/or pattern of isotopic labeling in the isotope-labeled free tubulin subunit(s) of the population(s) of microtubule molecules; i) measuring the molecular flux rates in molecular assemblage according to steps b) through i) in at least one living system not administered said one or more candidate agents; and j) comparing said molecular flux rates in the living system administered the candidate agent(s) to the molecular flux rates in the living system not administered the candidate agent(s). In another embodiment, this method may be used to evaluate the effect of an endogenous biological molecule on synaptic plasticity or synaptogenesis. It may also be used to measure synaptic plasticity or synaptogenesis, which includes steps b) through j) above. The methods of the present invention may also be used to measure the effect of a polynucleotide or gene on synaptic plasticity or synaptogenesis, which includes steps b) through j) above, wherein the comparing step j) is correlated with the expression level, structure of, presence or absence of the polynucleotide or gene.

The methods of the present invention may include the comparison of the molecular flux rates of the isotope-labeled tubulin subunit(s) incorporated into the population(s) of microtubule molecules to the molecular flux rates in the isotope-labeled free tubulin subunit(s). Alternatively, the molecular flux rates of the isotope-labeled tubulin subunit(s) incorporated into the population(s) of microtubule molecules may be compared to the molecular flux rates in body water or other metabolic precursor pools for free tubulin subunits.

In addition, the methods of the present invention may include the step of collecting from the living system one or more samples at known times or intervals after the step of administering the isotope-labeled substrate and after the step of exposing the living system to one or more candidate agents. The methods may also include the step of exposing the living system to combinations of two or more candidate agents.

In one aspect, the methods of the present invention include the isolation of microtubules and/or populations of microtubules based on their association with microtubule-associated proteins. In one embodiment, the microtubule-associated proteins include without limitation tau, Microtubule-Associated Protein2 (MAP2) and Stable Tubule Only Polypeptide (STOP).

In one embodiment, the effect on synaptic plasticity or synaptogenesis evaluated by the present invention is therapeutic to the living system. Alternatively, the effect may cause a toxic effect to the living system. The toxic effect may be a neurotoxic effect. In another embodiment, the effect may be a therapeutic effect. The therapeutic effect may be an increase in cognitive function and/or an improvement of at least one clinical sign or symptom of a cognitive disorder. In another embodiment, the effect may be measured in response to a specific dose or a range of doses of one or more candidate agents.

In another embodiment, the isotope used in the methods is a stable isotope. The stable isotope may be ²H. Alternatively, it may be a radioactive isotope, including without limitation ³H.

In other embodiments, the isotope-labeled substrate may be stable isotope-labeled water, which may include without limitation ²H₂O and H₂¹⁸O. The substrate may also include without limitation ²H₂O, ³H₂O, and an amino acid or precursor thereof. The isotope labeled substrate may also include without limitation ²H-labeled amino acids, ¹³C-labeled amino acids, ¹⁵N-labeled amino acids, ¹⁸O-labeled amino acids, ³H-labeled amino acids, ¹⁴C-labeled amino acids, and ³⁵S-labeled amino acids.

In one embodiment, one or more candidate agents of the methods of the present invention is an already-approved drug, including without limitation a Federal Food and Drug Administration-approved drug. Alternatively, the candidate agent(s) may be a new chemical entity or a biological factor.

The present invention provides a living system for evaluating an effect on synaptic plasticity or synaptogenesis. The living system may include without limitation eukaryotic cells, cell lines, cell cultures, isolated tissue preparations, rabbits, dogs, mice, rats, guinea pigs, pigs non-human primates, and humans. The living system may also be a human.

In one aspect, the present invention provides an isotopically-perturbed microtubule molecule generated by the method described herein, such as for example, the method including steps a) through j) described above.

In another aspect, the present invention provides kits for screening one or more compounds for an effect on synaptic plasticity or synaptogenesis according to the methods described herein. In one embodiment, the kit includes one or more isotope-labeled precursors and instructions for use of the kit. The kit may also include a tool for administration of precursor molecules and/or an instrument for collecting a sample from the subject.

In another aspect, the methods of the present invention also include the step of manufacturing one or more candidate agents at least partially identified by the methods described herein. Alternatively, the methods include the step of developing one or more candidate agents at least partially identified by the methods described herein. The developing step may be include the use of data obtained by the methods described herein. In one embodiment, the present invention provides a method including the steps of measuring the effect on synaptic plasticity or synaptogenesis according to the methods described herein, comparing the results of the step of exposing a living system to one or more candidate agents to the results of measuring the effect on synaptic plasticity or synaptogenesis in the presence of one or more candidate agents, determining whether one or more candidate agents changes the effect, and if it does, developing the agent. The agent may be therapeutic or diagnostic in nature. This method may further include the step of distributing the agent in commerce. It may also include the step of selling the agent.

In another aspect, the present invention provides a method for evaluating the effect of a candidate agent on synaptic connectivity, which may include the steps of a) exposing a test system to at least one candidate agent; b) administering at least one isotope-labeled substrate to the test system for a period of time sufficient for the isotope-labeled substrate to be incorporated into at least one tubulin subunit during formation of a microtubule population; c) obtaining from the test system a first sample having at least one isotope-labeled tubulin subunit incorporated into a first microtubule population at a first time period and a second sample having at least one isotope-labeled free tubulin subunit at the first time period; d) quantifying a test isotopic incorporation of the isotope-labeled tubulin subunit in the first microtubule population and a test isotopic incorporation of the isotope-labeled free tubulin subunit; e) providing the quantification of control isotopic incorporation of at least one isotope-labeled tubulin subunit incorporated into a microtubule population from the first time period and of at least one isotope-labeled free tubulin subunit from the first time period; f) comparing the test and control isotopic incorporations to determine an effect of the candidate agent. In other embodiments, the quantification step d) may include measuring one or more of the following:

1) the content of isotopic incorporation of both the isotope-labeled tubulin subunit incorporated into the microtubule population from the first time period and the isotope-labeled free tubulin subunit from the first time period;

2) the rate of isotopic incorporation of both the isotope-labeled tubulin subunit incorporated into the microtubule population from the first time period and the isotope-labeled free tubulin subunit from the first time period;

3) the pattern of isotopic incorporation of both the isotope-labeled tubulin subunit incorporated into the microtubule population from the first time period and the isotope-labeled free tubulin subunit from the first time period;

4) the rate of change in content of isotopic incorporation of both the isotope-labeled tubulin subunit incorporated into the microtubule population from the first time period and the isotope-labeled free tubulin subunit from the first time period; and

5) the rate of change in pattern of isotopic incorporation of both the isotope-labeled tubulin subunit incorporated into the microtubule population from the first time period and the isotope-labeled free tubulin subunit from the first time period.

In another embodiment, the comparing step f) also includes comparing molecular flux rates in the isotope-labeled tubulin subunit incorporated into the first microtubule population with molecular flux rates in the isotope-labeled free tubulin subunit. Alternatively, the comparing step f) includes comparing molecular flux rates in the isotope-labeled tubulin subunit incorporated into the first microtubule population with molecular flux rates in at least one metabolic precursor pool for free tubulin subunits. The precursor pool may be body water or it may includes at least one amino acid precursor.

In another embodiment, the isotopes of the methods of the present invention are stable isotopes. The isotope-labeled substrates of the methods may include without limitation stable isotope-labeled water, amino acid precursors, and amino acids. The label may be a stable isotope or a radioisotope. In one embodiment, the isotope-labeled substrate is an amino acid, including without limitation ²H-labeled amino acids, ¹³C-labeled amino acids, ¹⁵N-labeled amino acids, ¹⁸O-labeled amino acids, ³H-labeled amino acids, ¹⁴C-labeled amino acids, and ³⁵S-labeled amino acids.

In some embodiments, the methods of the invention include exposing the test system to a second candidate agent. In addition, the methods may also include exposing the test system to at least one specific dose of a candidate agent. The method may also include exposing the test system a second dose of the candidate agent. Additional doses of additional candidate agents are also possible.

In one aspect, the step of obtaining c) includes contacting the samples with a microtubule-associated protein binding agent. Suitable microtubule-associate proteins include without limitation tau, Microtubule-Associated Protein2 (MAP2) and Stable Tubule Only Polypeptide (STOP). The binding agent may be an antibody to a microtubule-associated protein.

In other embodiments, the effect on synaptic connectivity evaluated by the methods described herein is a therapeutic effect. Possible therapeutic effects include without limitation an increase of cognitive function and an improvement of at least one clinical sign or symptom of a cognitive disorder.

In one other aspect, the present invention provides kits for screening compounds for effects on synaptic connectivity according to the methods described herein. In one embodiment, the kits include at least one isotope-labeled substrate, and instructions for use of said kit. Additional kit components include without limitation a tool for administration of said substrate and an instrument for collecting a sample from a subject.

### BRIEF DESCRIPTION OF THE DRAWINGS

FIGS. 1A and 1B depict pathways of labeled hydrogen (²H or ³H) exchange from isotope-labeled water into selected free amino acids. Two NEAA's (alanine, glycine) and an EAA (leucine) are shown, by way of example. Alanine and glycine are presented in FIG. 1A. Leucine is presented in FIG. 1B. Abbreviations: TA, transaminase; PEP-CK, phosphoenolpyruvate carboxykinase; TCAC, tricarboxylic acid cycle. FIG. 1C depicts 18O-labeling of free amino acids by H218O for protein synthesis.

FIG. 2 shows the incorporation of ²H-labeled tubulin dimers into microtubule polymers.

FIG. 3 depicts the in vivo incorporation and exchange of tubulin dimers into microtubules (MT) in mouse brain. (A) Schematic representation of the strategy for isolating neuronal MT from different compartments. (B) Anti-tau and anti-MAP2 Western blots of cytosolic extract (lane 1), tau-nonassociated (lane 2), and tau-associated (lane 3) MT fractions, separated over anti-tau columns, showing quantitative capture of tau-associated MTs; MAP2-associated MTs are in the unbound fraction. (C) Kinetics of ²H incorporation from heavy water (²H₂O) into tubulin dimers and different MT fractions. Mice (n=3 per group) were labeled with 5% ²H₂O in body water for various times up to 24 hours, brains were dissected, and MT populations, isolated as in (A), were hydrolyzed. ²H incorporation into C—H bonds of alanine was measured by NCI-GC/MS and expressed as fractional synthesis (% newly synthesized during the labeling period; mean±SD). Single-exponential curve fits are shown. Plateaus values were reached in vivo for all fractions within 24 hours, leveling off at ca. 20% newly synthesized tubulin dimers, and half or one-third of this value, respectively, for tau-associated (growth cone) and MAP2/STOP-associated (somatodendritic and axonal shaft) MTs. The half-lives of the microtubule fraction, however, were not substantially different from those of free dimer.

FIG. 4 depicts the measurement of microtubule (MT) dynamics during neuronal maturation. (A and B) Postmitotic NT2-N neuronal cells were labeled continuously with 5% ²H₂O during culture with 10 ng/ml BDNF. Tubulin and MT fractions were analyzed as in FIG. 3. Outgrowth of neurites at day 5 (black arrows in (A)) and axonal differentiation at day 15 (white arrow in (B)) were visible by phase contrast microscopy (upper panels; scale bar, 10 □m). Essentially complete equilibration between ²H-labeled tubulin dimers with MT in different compartments was observed (lower panels). (C) After 7 weeks of culture with BDNF, NT2-N cells had established firm synaptic connections (upper panel); ²H₂O labeling for the last 24 hours of culture (lower panel) showed low-level incorporation of tubulin into MT (slightly greater in tau-associated MTs), resembling that in adult mouse brain (cf. FIG. 3C). All graphs show mean±SD for 3 culture dishes per condition.

FIG. 5 shows tubulin incorporation into dendritic and axonal microtubules (MT), both in cell culture and in vivo. Alternative methods to isolate distinct microtubule subpopulations and measure their dynamics are shown in (A, D). Baseline patterns of ²H incorporation into tubulin dimers and different MT subpopulations (mean±SD, n=3 mice) after 24 hours of ²H₂O labeling in vitro and in vivo (B, C and E, F). Cultured primary rat hippocampal neurons (B and E) and in vivo labeled mouse hippocampal tissue (C and F) were analyzed. STOP-associated MTs were isolated either as the tau and MAP2-unbound fraction (unbound MTs, (A-C)) or as the cold stable fraction (CS, (D-F)). In all cases, incorporation of tubulin into MTs was highest for tau-associated, intermediate for MAP2-associated, and lowest for STOP-associated MTs (unbound or CS; less than 3%).

FIG. 6 shows the effects of glutamate and a cAMP antagonist on MT dynamics during synaptic plasticity in vivo. In (A) and (B), mice received ICVC infusions of 6 μl water alone or containing 0.48 nmol glutamate (80 μM in the infusate), or glutamate plus 0.66 pmol Rp-cAMP (110 nM in the infusate). Animals were then labeled with ²H₂O for 8 hours (A) or 24 hours (B). At sacrifice, the hippocampal post-nuclear supernatant was fractionated as show in FIG. 5A; a separate aliquot was used for isolation of cold stable (CS) MTs (FIG. 5D). Glutamate injection stimulated label incorporation into new MAP2/STOP-MTs at 8 and 24 hours, and CS MTs at 24 hours; both were blocked by Rp-cAMP. In order to correct the effects of pharmacologic intervention on MT dynamics for the confounding changes in tubulin synthesis, the numbers above each bar express ²H label incorporation into MT as a percentage of ²H labeling in the corresponding tubulin dimer fractions. (C) Dose dependence of glutamate effects. ²H₂O labeling was initiated at time zero, 6 μl ICVC infusions with the indicated concentrations of glutamate were performed at 24 hours, and labeling was continued until sacrifice at 48 hours (24 hours post glutamate infusion). Hippocampal MTs were fractionated into tau-associated, MAP2-associated, and non-associated (STOP-associated) MT fractions, as in FIG. 5A. In all experiments, bars represent % new tubulin (mean±SD, n=3).

FIG. 7 depicts the effects of ICV glutamate on the relative amounts of MT subpopulations and on total protein synthesis in murine hippocampus. (A, B) Tubulin abundances are measured by Western blot analysis for α-tubulin at 8 hour (A) and 24 hours (B). Subpopulations were total extract (total), free tubulin dimers, tau-associated MTs, tau-nonassociated (MAP2/STOP) MTs and cold stable (CS) MTs. Mock and glutamate infused ICVC mice are shown (cf. FIGS. 6A and B). At 24 hours, treatment with 0.48 nmol glutamate increased the abundance of MAP2/STOP-MTs and CS-MTs (p<0.001) whereas the abundance of tau-MTs remained unchanged. (C) Fractional synthesis of total hippocampal proteins. Glutamate increased protein synthesis versus mock-treated animals (*p<0.01); this effect was blocked by coadministration with 0.66 pmol of Rp-cAMP (**p<0.02).

FIG. 8 illustrates uses of the inventions herein in a drug discovery process.

FIG. 9 is a schematic diagram showing the drug discovery, development, and approval (DDDA) process using effects on synaptic plasticity and synaptogenesis (i.e., data collected by the methods of the present invention) as a means for deciding to continue or cease efforts.

FIG. 10 depicts microtubule/tubulin exchange during hippocampus-dependent contextual memory. (A) Protocol for measurement of microtubule/tubulin exchange during contextual fear conditioning (CFC). Tubulin dimers and microtubules subsets were labeled to plateau through continuous ²H₂O labeling, starting one day before CFC training. Labeling was continued throughout CFC training (Day 2) and the contextual conditioning test until mice were euthanized (Day 3). Vehicle or nocodazole treated-mice were injected 4 hours before CFC training, consisting of three pairings of conditioned and unconditioned stimuli inside an observation chamber, as described in Example 7 below. (B) CFC experiments were conducted in groups of naïve, vehicle- (50% cyclodextrin) and nocodazole-treated (0.2 mg/kg) mice (mean±SD, n=8 per each group), respectively. Freezing responses, expressed as % of freezing to context, in the vehicle-treated mice were significantly higher in the naïve group (*p<0.001; one-way ANOVA). In contrast, the percent of freezing to context was significantly lower in nocodazole-treated mice as compared to the vehicle-treated mice (**p<0.001; one-way ANOVA). (C-E) ²H label incorporation into hippocampal tubulin dimers and microtubule subsets was compared at sacrifice in the indicated experimental groups. Microtubule subpopulations were fractionated as described below, and the percentage of newly synthesized tubulin was quantified by GC/MS (mean±SD, n=3). (C) and (D) In vehicle-CFC trained mice, the increased freezing response corresponded with an increase in ²H labeling of MAP2- and CS-associated MTs, compared to naïve control animals (§p<0.01 and §§p<0.05; two-way ANOVA). (E) The nocodazole-induced amnesia correlated with a significant disassembly of MAP2- and tau-associated MTs, as compared to vehicle-treated animals (# p<0.001; two-way ANOVA). In (C-E), bars represent % new tubulin (mean±SD, n=3).

### DETAILED DESCRIPTION OF THE INVENTION

### I. Introduction

### A. Overview of the Invention

The adult brain is characterized by remarkable functional plasticity, which allows learning and, thus, behavioral adaptation, as well as retention of what has been learned (memory). At the neuroanatomic level, functional plasticity is reflected in new and/or strengthened connections among neurons. Neuronal connectivity occurs primarily via synapses. Synaptic plasticity therefore represents the physical or biochemical substrate upon which learning and memory are based in the brain.

Disorders of cognition are typically characterized by impaired addition of new information (learning) or retention of information (memory). These disorders are believed to reflect alterations in the neurobiological processing of information, and therefore to be mediated biochemically by events related to synaptic plasticity. Disorders of learning and memory (cognitive disorders) include Alzheimer's disease and other dementias, learning disabilities, traumatic brain injury, cerebrovascular accident, various psychiatric conditions (e.g., schizophrenia) and other conditions. Cognitive disorders are responsible for enormous morbidity as well as premature mortality, in the United States and world-wide.

At present, there exist no objective laboratory-based metrics of synaptic plasticity in the living brain. Current assays of neuronal connectivity in living animals consist primarily of behavioral tests, as well as electrophysiologic measures and other global, non-biochemical techniques. Behavioral measures are widely recognized to be fundamentally limited in terms of reproducibility, sensitivity, precision and capacity for biological targeting to specific brain regions or molecular targets. Behavioral tests also typically require large numbers of animals, to observe significant differences between groups in response to experimental manipulations. Particular characteristics of synaptic plasticity, such as long-term potentiation (the strengthening effect on synaptic connections and learning induced by repeated exposures to a stimulus) remain particularly difficult to measure quantitatively or monitor in the living organism.

Accordingly, there is a long-recognized need in the field of cognitive science and brain plasticity for an objective, biochemical marker (biomarker) of synaptic connectivity, including synaptogenesis and synaptic plasticity in the living brain. The capacity to detect and quantify a “biochemical record” of new neuronal connections and their maintenance or loss during a period of time in the mature brain would represent a fundamental advance in this field. The ability to directly study, in a reproducible manner, the influence of environmental factors (e.g., enriched environment, different learning paradigms), genetics (e.g., strain differences), physiologic mediators (e.g., neurotransmitters, hormones) and pharmacologic agents (e.g., drugs to enhance learning or memory), on synaptic plasticity, long-term potentiation or other biochemical aspects of adaptation, learning, memory and general cognition, would be a major advance. A metric of this type could be correlated with functional (e.g., cognitive, behavioral) outcomes and, ultimately, replace the latter. Of particular value would be the capacity to discover drugs that favorably modulate cognitive function and to discard candidate drugs that unfavorably alter cognitive function.

The Applicants disclose here an invention that allows synaptic plasticity and, therefore, the biochemical events underlying learning and memory, to be measured directly and in a reproducible, high-throughput manner in the brain of living animals. The Applicants have discovered that the dynamics of brain microtubules can me measured in vivo by a stable isotope-mass spectrometric labeling approach, and that the dynamics of specific microtubule fractions, isolated biochemically from the brain, reveal the dynamics of synaptic plasticity and neuronal connectivity in vivo. Moreover, the dynamics of brain microtubules correlate with not only neurochemical influences, but also with art-accepted behavioral measures in living animals.

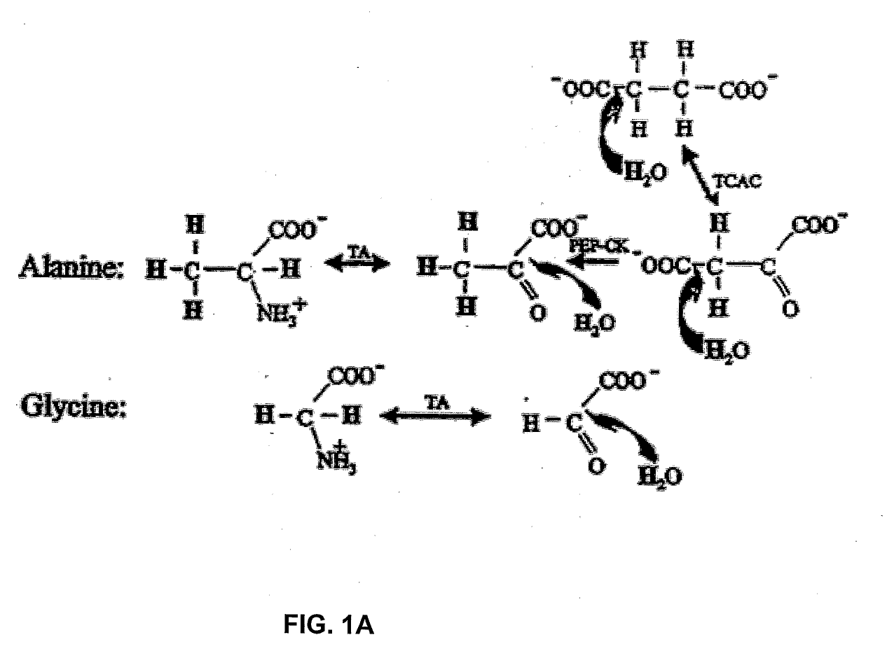

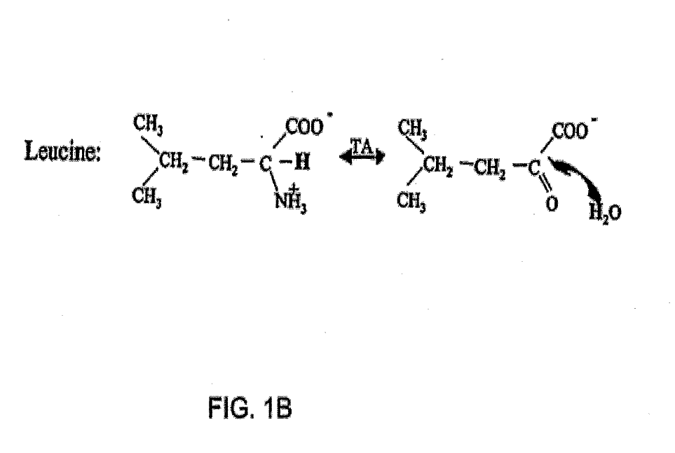

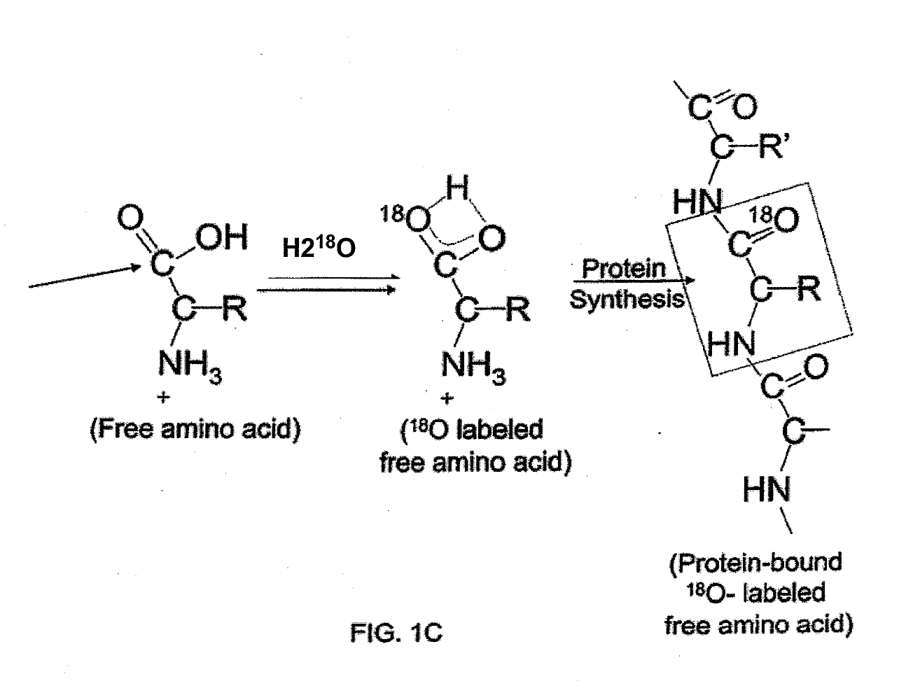

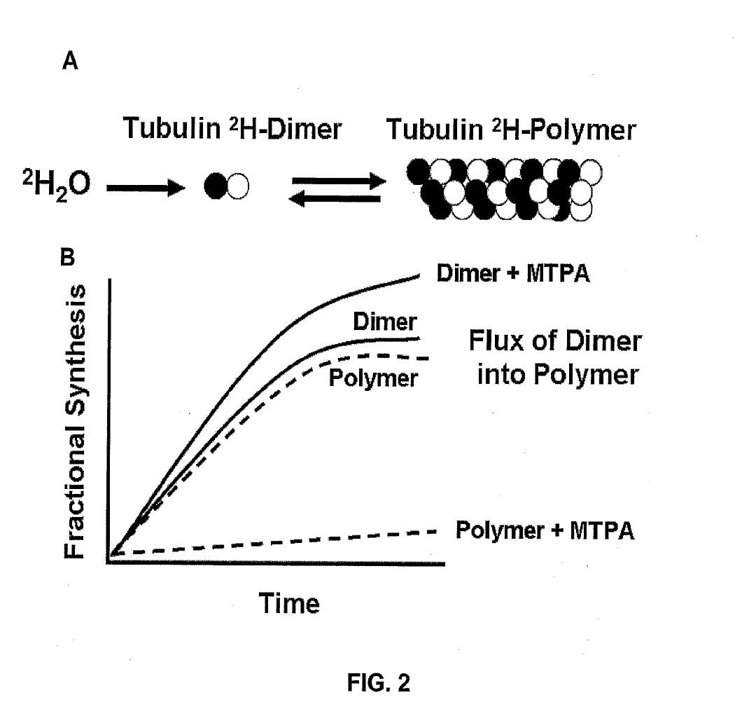

The methods of the present invention make use of deuterated water (²H₂O) to isotopically label free tubulin subunits (see FIGS. 1A, 1B). The skilled artisan will appreciate that other isotopes may be used and may be administered via labeled amino acids or other precursors of protein biosynthesis as described more fully, infra (also see FIG. 1C). The ²H label enters newly synthesized free tubulin subunits via metabolic pathways for the biosynthesis of nonessential amino acids. Incorporation of ²H into newly synthesized free tubulin subunits appears in the tubulin dimer pool before being incorporated through polymerization into microtubules (see FIG. 2A). In biological settings in which microtubules are highly dynamic, ²H-label accumulates in the dimer and polymer pools at similar rates reflecting rapid exchange kinetics between the two pools (i.e., dynamic instability of the microtubules). In this dynamic state ²H label accumulates in polymers at about the same rate as it appears in dimers. However, in settings where microtubules are less dynamic or stabilized by microtubule-targeted tubulin-polymerizing agents (MTPAs) or by endogenous microtubule-stabilizing factors, ²H-label in newly synthesized tubulin appears in the dimer pool at a rate proportional to the biosynthetic rate of free tubulin, whereas incorporation into microtubule polymers is slower and diminished, often dramatically so (see FIG. 2B).

These discoveries and the invention disclosed herein therefore enable an objective biochemical record of neuronal connectivity (synaptic plasticity) in the living brain to be generated and monitored by research scientists. A particularly valuable consequence of the invention disclosed herein is the capacity to screen for, select and discard drug candidates that modulate synaptic plasticity and therefore may improve (or worsen) learning, memory, or other aspects of cognitive function. This drug screening and filtering approach for modulators of learning and memory is demonstrated to be capable of high-throughput in vivo in animal models.

### B. Biochemistry and Cell Biology of the Nervous System

Neurons are a unique cell type in that they contain long processes (neurites) which cover more than 99% of the cellular volume. This requires the presence of a sophisticated molecular machinery in order to establish and maintain their specialized morphology. The primary molecular machinery responsible for the cellular integrity of the neuron is the microtubule framework. Microtubules are very abundant in neurons where they facilitate the formation of, and confer stability to, neurites (axons and dendrites). The state of stability and dynamic instability of axonal microtubules (“microtubule dynamics”) represent a signaling pathway within neurons. The assembly of microtubules is regulated largely by microtubule-associated proteins (MAPs). The neuronal MAPs have a specific polar distribution. For example, tau is localized only in the axonal compartment and it is involved in neurodegenerative disorders. Tau influences microtubule assembly, neurite outgrowth and neuritic stability. This is accomplished through tau's ability to regulate the highly dynamic behavior of microtubules and thus stabilize them. Therefore, the integrity of the microtubule structure serves as a biosensor of the normal homeostasis in neurons, and any disruption in the regulation of that integrity can lead to the activation of cellular stress responses.

Over the past decade, the brain has been recognized as a dynamic system whose response to stimuli through electrical and neurochemical circuitry also relies on specific biochemical and structural changes. Clearly, rearrangements of the microtubule cytoskeleton are of fundamental importance for the establishment of neural circuits during brain development and for synaptic plasticity in mature neurons. Understanding the disruption of synaptic connectivity in neurodegenerative disorders, and correcting these defects by pharmacologic or regenerative approaches, will require an appreciation of the underlying dynamics of compartmentalized microtubules. The kinetic measurement techniques described here may be useful as a biochemical record of synaptic plasticity and neuronal maturation and may ultimately prove valuable for correlation with behavioral measures, such as learning and memory.

### II. General Techniques

The practice of the present invention will employ, unless otherwise indicated, conventional techniques of molecular biology (including recombinant techniques), neurobiology, basic neuroscience, clinical neuroscience and neurology, microbiology, cell biology, biochemistry, immunology and behavioral biology (including learning and memory techniques), which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); and Mass isotopomer distribution analysis at eight years: theoretical, analytic and experimental considerations by Hellerstein and Neese (Am J Physiol 276 (Endocrinol Metab. 39) E1146-E1162, 1999). Furthermore, procedures employing commercially available assay kits and reagents will typically be used according to manufacturer-defined protocols unless otherwise noted.

### III. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, Mass isotopomer distribution analysis at eight years: theoretical, analytic and experimental considerations by Hellerstein and Neese (Am J Physiol 276 (Endocrinol Metab. 39) E1146-E1162, 1999). As appropriate, procedures involving the use of commercially available kits and reagents are generally carried out in accordance with manufacturer defined protocols and/or parameters unless otherwise noted.

“Molecular flux rates” or “flux” refers to the rate of synthesis and/or breakdown of molecules within a cell, tissue, or organism. “Molecular flux rates” also refers to a molecule's input into or removal from a pool of molecules, and is therefore synonymous with the flow into and out of said pool of molecules.

“Metabolic pathway” refers to any linked series of two or more biochemical steps in a living system (i.e., a biochemical process), the net result of which is a chemical, spatial or physical transformation of a molecule or molecules. Metabolic pathways are defined by the direction and flow of molecules through the biochemical steps that comprise the pathway. Molecules within metabolic pathways can be of any biochemical class, e.g., including but not limited to lipids, proteins, amino acids, carbohydrates, nucleic acids, polynucleotides, porphyrins, glycosaminoglycans, glycolipids, intermediary metabolites, inorganic minerals, ions, etc.

“Flux rate through a metabolic pathway” refers to the rate of molecular transformations through a defined metabolic pathway. The unit of flux rates through pathways is chemical mass per time (e.g., moles per minute, grams per hour). Flux rate through a pathway optimally refers to the transformation rate from a clearly defined biochemical starting point to a clearly defined biochemical end-point, including all the stages in between in the defined metabolic pathway of interest.

“Isotopes” refer to atoms with the same number of protons and hence of the same element but with different numbers of neutrons (e.g., ¹H vs. ²H or D).

“Isotopologues” refer to isotopic homologues or molecular species that have identical elemental and chemical compositions but differ in isotopic content (e.g., CH₃NH₂ vs. CH₃NHD in the example above). Isotopologues are defined by their isotopic composition therefore each isotopologue has a unique exact mass but may not have a unique structure. An isotopologue is usually comprised of a family of isotopic isomers (isotopomers) which differ by the location of the isotopes on the molecule (e.g., CH₃NHD and CH₂DNH₂ are the same isotopologue but are different isotopomers).

“Isotope-labeled water” includes water labeled with one or more specific heavy isotopes of either hydrogen or oxygen. Specific examples of isotope-labeled water include ²H₂O, ³H₂O, and H₂ ¹⁸O.

“Chemical entity” includes any molecule, chemical, or compound, whether new or known, that is administered to a living system for the purpose of screening it for biological or biochemical activity toward the goal of discovering potential therapeutic agents (drugs or drug candidates or drug leads) or uncovering toxic effects (industrial chemicals, pesticides, herbicides, food additives, cosmetics, and the like).

“Drug leads” or “drug candidates” are herein defined as chemical entities or biological molecules that are being evaluated as potential therapeutic agents (drugs). “Drug agents” or “agents or “compounds” are used interchangeably herein and describe any composition of matter (e.g., chemical entity or biological factor) that is administered, approved or under testing as potential therapeutic agent or is a known therapeutic agent.

“Known drugs” or “known drug agents” or “already-approved drugs” refers to agents (i.e., chemical entities or biological factors) that have been approved for therapeutic use as drugs in human beings or animals in the United States or other jurisdictions. In the context of the present invention, the term “already-approved drug” means a drug having approval for an indication distinct from an indication being tested for by use of the methods disclosed herein. Using psoriasis and fluoxetine as an example, the methods of the present invention allow one to test fluoxetine, a drug approved by the FDA (and other jurisdictions) for the treatment of depression, for effects on biomarkers of psoriasis (e.g., keratinocyte proliferation or keratin synthesis); treating psoriasis with fluoxetine is an indication not approved by FDA or other jurisdictions. In this manner, one can find new uses (in this example, anti-psoriatic effects) for an already-approved drug (in this example, fluoxetine).

“Biological factor” refers to a compound or compounds made by living organisms having biological or physiological activities (e.g., preventive, therapeutic and/or toxic effects). Examples of biological factors include, but are not limited to, vaccines, polyclonal or monoclonal antibodies, recombinant proteins, isolated proteins, soluble receptors, gene therapy products, and the like. As used herein, the term “biologics” is synonymous with “biological factor.”

“Compound” means, in the context of the present invention, any new chemical entity, chemical entity, drug lead, drug candidate, drug, drug agent, agent, known drug, known drug agent, already-approved drug, biologic, or biological factor. The term is meant to encompass all chemical and biological molecules.

“Food additive” includes, but is not limited to, organoleptic agents (i.e., those agents conferring flavor, texture, aroma, and color), preservatives such as nitrosamines, nitrosamides, N-nitroso substances and the like, congealants, emulsifiers, dispersants, fumigants, humectants, oxidizing and reducing agents, propellants, sequestrants, solvents, surface-acting agents, surface-finishing agents, synergists, pesticides, chlorinated organic compounds, any chemical ingested by a food animal or taken up by a food plant, and any chemical leaching into (or otherwise finding its way into) food or drink from packaging material. The term is meant to encompass those chemicals which are added into food or drink products at some step in the manufacturing and packaging process, or find their way into food by ingestion by food animals or uptake by food plants, or through microbial byproducts such as endotoxins and exotoxins (pre-formed toxins such as botulinin toxin or aflatoxin), or through the cooking process (such as heterocyclic amines, e.g., 2-amino-3-methyllimidazo[4,5-f]quinolone), or by leaching or some other process from packaging material during manufacturing, packaging, storage, and handling activities.

“Industrial chemical” includes, but is not limited to, volatile organic compounds, semi-volatile organic compounds, cleaners, solvents, thinners, mixers, metallic compounds, metals, organometals, metalloids, substituted and non-substituted aliphatic and acyclic hydrocarbons such as hexane, substituted and non-substituted aromatic hydrocarbons such as benzene and styrene, halogenated hydrocarbons such as vinyl chloride, aminoderivatives and nitroderivatives such as nitrobenzene, glycols and derivatives such as propylene glycol, ketones such as cyclohexanone, aldehydes such as furfural, amides and anhydrides such as acrylamide, phenols, cyanides and nitriles, isocyanates, and pesticides, herbicides, rodenticides, and fungicides.

“Environmental pollutant” includes any chemical not found in nature or chemicals that are found in nature but artificially concentrated to levels exceeding those found in nature (at least found in accessible media in nature). So, for example, environmental pollutants can include any of the non-natural chemicals identified as an occupational or industrial chemical yet found in a non-occupational or industrial setting such as a park, school, or playground. Alternatively, environmental pollutants may comprise naturally occurring chemicals such as lead but at levels exceeding background (for example, lead found in the soil along highways deposited by the exhaust from the burning of leaded gasoline in automobiles). Environmental pollutants may be from a point source such as a factory smokestack or industrial liquid discharge into surface or groundwater, or from a non-point source such as the exhaust from cars traveling along a highway, the diesel exhaust (and all that it contains) from buses traveling along city streets, or pesticides deposited in soil from airborne dust originating in farmlands. As used herein, “environmental contaminant” is synonymous with “environmental pollutant.”

“Living system” includes, but is not limited to, cells, cell lines, animal models of disease, guinea pigs, rabbits, dogs, cats, other pet animals, mice, rats, non-human primates, and humans. A living system includes a “test system”, which includes, but is not limited to, vertebrates, including animals, particularly mammals, and particularly human. Included are animals having a variety of disease states, including learning and memory disorders. According to the present invention, at least one candidate agent is administered to a test system. A control system may be a system that is not administered a candidate agent or a system that is disease-free.

A “biological sample”, “sample”, or grammatical equivalents thereof encompasses any sample obtained from a cell, tissue, or organism. The definition encompasses blood and other liquid samples of biological origin, that are accessible from an organism through sampling by invasive means (e.g., surgery, open biopsy, endoscopic biopsy, and other procedures involving non-negligible risk) or by minimally invasive or non-invasive approaches (e.g., urine collection, blood drawing, needle aspiration, and other procedures involving minimal risk, discomfort or effort). The definition also includes samples that have been manipulated in any way after their procurement, such as by treatment with reagents, solubilization, or enrichment for certain components, such as proteins or organic metabolites. The term “biological sample” also encompasses a clinical sample such as serum, plasma, other biological fluid, or tissue samples, and also includes cells in culture, cell supernatants and cell lysates.

“Biological fluid” refers, but is not limited to, urine, blood, interstitial fluid, edema fluid, saliva, lacrimal fluid, inflammatory exudates, synovial fluid, abscess, empyema or other infected fluid, cerebrospinal fluid, sweat, pulmonary secretions (sputum), seminal fluid, feces, bile, intestinal secretions, or other biological fluid.

“Exact mass” refers to mass calculated by summing the exact masses of all the isotopes in the formula of a molecule (e.g., 32.04847 for CH₃NHD).

“Nominal mass” refers to the integer mass obtained by rounding the exact mass of a molecule.

“Mass isotopomer” refers to family of isotopic isomers that is grouped on the basis of nominal mass rather than isotopic composition. A mass isotopomer may comprise molecules of different isotopic compositions, unlike an isotopologue (e.g., CH₃NHD, ¹³CH₃NH₂, CH₃¹⁵NH₂ are part of the same mass isotopomer but are different isotopologues). In operational terms, a mass isotopomer is a family of isotopologues that are not resolved by a mass spectrometer. For quadrupole mass spectrometers, this typically means that mass isotopomers are families of isotopologues that share a nominal mass. Thus, the isotopologues CH₃NH₂ and CH₃NHD differ in nominal mass and are distinguished as being different mass isotopomers, but the isotopologues CH₃NHD, CH₂DNH₂, ¹³CH₃NH₂, and CH₃¹⁵NH₂ are all of the same nominal mass and hence are the same mass isotopomers. Each mass isotopomer is therefore typically composed of more than one isotopologue and has more than one exact mass. The distinction between isotopologues and mass isotopomers is useful in practice because all individual isotopologues are not resolved using quadrupole mass spectrometers and may not be resolved even using mass spectrometers that produce higher mass resolution, so that calculations from mass spectrometric data must be performed on the abundances of mass isotopomers rather than isotopologues. The mass isotopomer lowest in mass is represented as M₀; for most organic molecules, this is the species containing all ¹²C, ¹H, ¹⁶O, ¹⁴N, etc. Other mass isotopomers are distinguished by their mass differences from M₀ (M₁, M₂, etc.). For a given mass isotopomer, the location or position of isotopes within the molecule is not specified and may vary (i.e., “positional isotopomers” are not distinguished).

“Mass isotopomer envelope” refers to the set of mass isotopomers comprising the family associated with each molecule or ion fragment monitored.

“Mass isotopomer pattern” refers to a histogram of the abundances of the mass isotopomers of a molecule. Traditionally, the pattern is presented as percent relative abundances where all of the abundances are normalized to that of the most abundant mass isotopomer; the most abundant isotopomer is said to be 100%. The preferred form for applications involving probability analysis, such as mass isotopomer distribution analysis (MIDA), however, is proportion or fractional abundance, where the fraction that each species contributes to the total abundance is used. The term “isotope pattern” may be used synonomously with the term “mass isotopomer pattern.”

“Monoisotopic mass” refers to the exact mass of the molecular species that contains all ¹H, ¹²C, ¹⁴N, ¹⁶O, ³²S, etc. For isotopologues composed of C, H, N, O, P, S, F, Cl, Br, and I, the isotopic composition of the isotopologue with the lowest mass is unique and unambiguous because the most abundant isotopes of these elements are also the lowest in mass. The monoisotopic mass is abbreviated as m₀ and the masses of other mass isotopomers are identified by their mass differences from m₀ (m₁, m₂, etc.).

“Isotopically perturbed” refers to the state of an element or molecule that results from the explicit incorporation of an element or molecule with a distribution of isotopes that differs from the distribution that is most commonly found in nature, whether a naturally less abundant isotope is present in excess (enriched) or in deficit (depleted).

By “molecule of interest” is meant any molecule (polymer and/or monomer), including but not limited to, amino acids, carbohydrates, fatty acids, peptides, sugars, lipids, nucleic acids, polynucleotides, glycosaminoglycans, polypeptides, or proteins that are present within a metabolic pathway within a living system. In the context of the present invention, a “molecule of interest” may be a “biomarker” of disease and its flux rate, relative to the flux rate of an unexposed or otherwise healthy subject (i.e., control subject), may represent clinically non-observant or subtle pathophysiological occurrences in a subject of interest that may be predictive of future disease or injury in the subject of interest. In this manner, comparing the flux rates of one or more biomarkers of interest in a subject of interest with the flux rates of one or more biomarkers of interest in a control subject, will find use in diagnosing the subject of interest with, or evaluating or quantifying the subject of interest's risk in acquiring, a disease of interest. Moreover, such information will find use in establishing a prognosis for a subject of interest having a disease of interest, monitoring the progression of a disease of interest in a subject of interest, or evaluating the therapeutic efficacy of a treatment regimen in a subject of interest having a disease of interest.

By “subject of interest” is meant a human or animal having a disease of interest or having some level of risk in acquiring a disease of interest.

By “control subject” is meant a human or animal not having the disease of interest or not having some level of risk in acquiring the disease of interest.

“Monomer” refers to a chemical unit that combines during the synthesis of a polymer and which is present two or more times in the polymer.

“Polymer” refers to a molecule synthesized from and containing two or more repeats of a monomer. A “biopolymer” is a polymer synthesized by or in a living system or otherwise associated with a living system.

“Protein” refers to a polymer of amino acids. As used herein, a “protein” may refer to long amino acid polymers as well as short polymers such as peptides.

By “amino acid” is meant any amphoteric organic acid containing the amino group (i.e., NH₂). The term encompasses the twenty common (often referred in the art as “standard” or sometimes as “naturally occurring”) amino acids as well as the less common (often referred in the art as “nonstandard”) amino acids. Examples of the twenty common amino acids include the alpha-amino acids (or α-amino acids), which have the amino group in the alpha position, and generally have the formula RCH—(NH₂)—COOH. The α-amino acids are the monomeric building blocks of proteins and can be obtained from proteins through hydrolysis. Examples of nonstandard amino acids include, but are not limited to γ-aminobutyric acid, dopamine, histamine, thyroxine, citrulline, ornithine, homocysteine, and S-adenosylmethionine.

“Isotope labeled substrate” includes any isotope-labeled precursor molecule that is able to be incorporated into a molecule of interest in a living system. Examples of isotope labeled substrates include, but are not limited to, ²H₂O, ³H₂O, ²H-glucose, ²H-labeled amino acids, ²H-labeled organic molecules, ¹³C-labeled organic molecules, ¹⁴C-labeled organic molecules, ¹³CO₂, ¹⁴CO₂, ¹⁵N-labeled organic molecules and ¹⁵NH₃.

“Deuterated water” refers to water incorporating one or more ²H isotopes.

“Administer[ed]” includes a living system exposed to a chemical entity or entities. Such exposure can be from, but is not limited to, topical application, oral ingestion, inhalation, subcutaneous injection, intraperitoneal injection, intravenous injection, and intraarterial injection, in animals or other higher organisms.

By “toxic effect” is meant an adverse response by a living system to a candidate agent. A toxic effect in the context of the present invention may be learning impairment or memory impairment or even complete learning loss or memory loss.

An “individual” is a vertebrate, preferably a mammal, more preferably a human.

By “mammal” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

“At least partially identified” in the context of drug discovery and development means at least one clinically relevant pharmacological characteristic of a drug agent (i.e., a “compound”) has been identified using one or more of the methods of the present invention. This characteristic may be a desirable one, for example, increasing or decreasing molecular flux rates through a metabolic pathway that contributes to a disease process, altering signal transduction pathways or cell surface receptors that alter the activity of metabolic pathways relevant to a disease, inhibiting activation of an enzyme and the like. Alternatively, a pharmacological characteristic of a drug agent may be an undesirable one for example, the production of one or more toxic effects. There are a plethora of desirable and undesirable characteristics of drug agents well known to those skilled in the art and each will be viewed in the context of the particular drug agent being developed and the targeted disease. Of course, a drug agent can be more than at least partially identified when, for example, when several characteristics have been identified (desirable or undesirable or both) that are sufficient to support a particular milestone decision point along the drug development pathway. Such milestones include, but are not limited to, pre-clinical decisions for in vitro to in vivo transition, pre-IND filing go/no go decision, phase I to phase II transition, phase II to phase III transition, NDA filing, and FDA approval for marketing. Therefore, “at least partially” identified includes the identification of one or more pharmacological characteristics useful in evaluating a drug agent in the drug discovery/drug development process. A pharmacologist or physician or other researcher may evaluate all or a portion of the identified desirable and undesirable characteristics of a drug agent to establish its therapeutic index. This may be accomplished using procedures well known in the art.

“Manufacturing a drug agent” in the context of the present invention includes any means, well known to those skilled in the art, employed for the making of a drug agent product. Manufacturing processes include, but are not limited to, medicinal chemical synthesis (i.e., synthetic organic chemistry), combinatorial chemistry, biotechnology methods such as hybridoma monoclonal antibody production, recombinant DNA technology, and other techniques well known to the skilled artisan. Such a product may be a final drug agent that is marketed for therapeutic use, a component of a combination product that is marketed for therapeutic use, or any intermediate product used in the development of the final drug agent product, whether as part of a combination product or a single product. “Manufacturing drug agent” is synonymous with “manufacturing a compound.”

By “biomarker” is meant a biochemical measurement from the organism which is useful or potentially useful for measuring the initiation, progression, severity, pathology, aggressiveness, grade, activity, disability, mortality, morbidity, disease sub-classification or other underlying pathogenic or pathologic feature of one or more diseases. The concept of a biomarker also includes a physical measurement on the body, such as blood pressure, which is useful for measuring the initiation, progression, severity, pathology, aggressiveness, grade, activity, disability, mortality, morbidity, disease sub-classification or other underlying pathogenic or pathologic feature of one or more diseases. The concept of a biomarker also includes a pharmacological or physiological measurement which is used to predict a toxicity event in an animal or a human. A biomarker may be the target for monitoring the outcome of a therapeutic intervention (i.e., the target of a drug agent).

By “evaluate” or “evaluation” or “evaluating,” in the context of the present invention, is meant a process whereby the activity, toxicity, relative potency, potential therapeutic value and/or efficacy, significance, or worth of a chemical entity, biological factor, combination of chemical entities, or combination of biological factors is determined through appraisal and study, usually by means of comparing experimental outcomes to established standards and/or conditions. The term embraces the concept of providing sufficient information for a decision-maker to make a “go/no go” decision on a chemical entity or biological factor (or combinations of chemical entities or combinations of biological factors) to proceed further in the drug development process. A “go/no go” decision may be made at any point or milestone in the drug development process including, but not limited to, any stage within pre-clinical development, the pre-clinical to Investigational New Drug (IND) stage, the Phase I to Phase II stage, the Phase II to more advanced phases within Phase II (such as Phase IIb), the Phase II to Phase III stage, the Phase III to the New Drug Application (NDA) or Biologics License Application (BLA) stage, or stages beyond (such as Phase IV or other post-NDA or post-BLA stages). The term also embraces the concept of providing sufficient information to select “best-in-breed” (or “best-of-breed”) in a class of compounds (chemical entities, biologics).

By “characterize,” “characterizing,” or “characterization,” in the context of the present invention is meant an effort to describe the character or quality of a chemical entity or combination of chemical entities. As used herein, the term is nearly equivalent to “evaluate,” yet lacks the more refined aspects of “evaluate,” in which to “evaluate” a drug includes the ability to make a “go/no go” decision (based on an assessment of therapeutic value) on proceeding with that drug or chemical entity through the drug development process.

By “condition” or “medical condition” is meant the physical status of the body as a whole or of one of its parts. The term is usually used to indicate a change from a previous physical or mental status, or an abnormality not recognized by medical authorities as a disease or disorder. Examples of “conditions” or “medical conditions” include obesity and pregnancy.

By “axon” is meant a highly specialized relatively long extension (process) of a nerve cell that normally transmits outgoing signals (i.e., action potentials) from one cell body to another cell. Each nerve cell has one axon, which can be relatively short in the brain but can be up to several feet long in other parts of the body.

By “dendrite” is meant a slender, typically branched projection that extends from the cell bodies of neurons. Neurons may contain multiple dendrites, which are stimulated by neurotransmitters, receive impulses from the nerve fibers (axons) of other neurons, and convey them toward their nerve cell bodies.

By “neuronal differentiation” is meant the process by which pluripotent cells become progressively more specialized and mature into neurons.

By “axonal sprouting and branching” is meant the process by which an axon sprouts (occurring in the axonal growth cone) or branches (occurring in the axonal shaft) to connect with other nerve cells, forming new neural pathways.

By “synapse” is meant a specialized junction through which cells of the nervous system signal to one another and to non-neuronal cells such as muscles or glands.

By “synaptic connectivity” is meant synaptogenesis and/or synaptic plasticity.

By “synaptogenesis” is meant the process of creating synaptic connections.

By “synaptic plasticity” is meant the general process of modulation and adaptation of neuronal connections in the living brain in response to environmental and chemical influences. Synaptic plasticity is part of the Hebbian theory of the neurochemical foundation of memory and learning.

By “neurotransmitter” is meant a chemical substance that is used to relay, amplify and modulate electrical signals (action potentials) between a presynaptic and a postsynaptic neuron. Neurotransmitters are released from neurons, diffuse across the space between cells (synaptic cleft) and bind to receptors. Neurotransmitters may cause either excitatory or inhibitory post-synaptic effects.

By “neurotrophin” or “neurotrophic factor” is meant a chemical substance or molecule, any of a family of growth factors, that encourage survival of nervous tissue by preventing apoptosis in the neuron. Neurotrophic factors also promote neuronal differentiation and synaptogenesis in vitro. An example of a neurotrophic factor is brain-derived neurotrophic factor (BDNF).

As used herein, “glutamate” is a prominent excitatory neurotransmitter of the central nervous system (CNS).

By “neuronal microtubules” is meant a protein structure composed of polymers of tubulin, occurring singly, in pairs, triplets or bundles in living cells. Neuronal microtubules are present in different locations in neurons (soma, dendrites and axon) and in association with different proteins (e.g., tau, MAP2 and STOP). Microtubules are required to establish and maintain neuronal differentiation and long distance transport of neurotransmitter substances along the axons to distant synapses.

By “tubulin” is meant the principal protein component of microtubules. Tubulin is a dimer composed of two globular polypeptides, alpha-tubulin and beta-tubulin. Microtubules are assembled from dimers of alpha- and beta-tubulin (α- and β-tubulin).

“MAPs” or “microtubule-associated proteins” are proteins that, upon binding to a microtubule, alter its function and/or behavior.

As used herein, “tau” or “tau protein” is a major class of microtubule-associated proteins (MAPs) isolated from the brain. In nerve cells tau is highly enriched in the axonal growth cone. Tau proteins bind to several unpolymerized tubulin molecules simultaneously and speed up the nucleation process of tubulin polymerization in brain. Tau regulates the turnover/assembly of dynamic axonal growth cone microtubules. Chemically modified tau proteins also appear to be involved in the formation and/or composition of the neurofibrillary tangles and neuropil threads found in Alzheimer's disease.

As used herein, “MAP2” or “Microtubule-Associated Protein2” is a high molecular weight microtubule-associated protein that is highly enriched in neuronal dendritic microtubules. Under certain conditions, MAP2 is required for tubulin assembly into microtubules and stabilizes the assembled microtubules, regulating their dynamics.

As used herein, “STOP” or “Stable Tubule Only Polypeptide” is a neuronal Ca²⁺-calmodulin-regulated microtubule associated protein. STOP stabilizes microtubules indefinitely against in vitro disassembly induced by cold temperature, millimolar calcium or drugs.

By “neuronal cold-stable microtubules” is meant an abundant subpopulation of axonal microtubules that are stable to disassembly induced by both drugs and cold-temperature. Resistance to microtubule disassembly by drugs and cold-temperature is largely due to polymer association with STOP (stable-tubule-only-polypeptide) protein.

“Dynamics” refers to the kinetic features of a molecule or system of molecules. As used herein, it refers to chemical rates in the dimension of time (e.g., mass or moles per unit of time, for example moles per minute, grams per hour) and includes synthesis rates, breakdown rates, turnover rates, transformation rates, interchange rates, assembly and disassembly rates, polymerization and depolymerization rates, and other aspects of the kinetic behavior of microtubules. It should be noted that some of these rates can be related; e.g., the rate of synthesis and the rate of breakdown can be combined to give the turnover rate, etc. This is also referred to in some instances as “the flux rate through a metabolic pathway”. In some cases, in particular for non-reversible or very slow depolymerizations, “flux rate through a pathway” can refer to the transformation rate from a clearly defined biochemical starting point to a clearly defined biochemical endpoint.

By “learning” is meant the cognitive process of acquiring skill or knowledge.

By “memory” is meant the ability to process, or the act of remembering or recalling, especially the ability to reproduce what has been learned or explained. This process requires attention, storage, and retrieval. Duration of memory is a function of the number of repetitions of an experience. There are two forms of memory, short-term and long-term memory. Short-term memory does not require the synthesis of new protein. Long-term memory does require the synthesis of new protein.

By “LTP” or “long-term potentiation” is meant a sustained change in connection strength (potentiation), largely synaptic that follows some priming events, such as a barrage of impulses. LTP lasts for an extended period of time, minutes to hours in vitro and hours to days and months in vivo. LTP is thought to provide the physiological scaffolding for slowly making the anatomical changes that more permanently increase the synaptic strength (memory consolidation).

By “explicit memory” is meant memory that people hold near or dear. These memories require conscious recall and are concerned with memories of people, places, objects and events.

By “implicit memory” is meant memory of perceptual and motor skills that is expressed through performance without conscious recall of past episodes.

By “sensitization” is meant a form of learned fear in which a person or an experimental animal learns to respond strongly to an otherwise neutral stimulus. A single stimulus gives rise to memory that can last for hours; four to five stimuli can last for several days.

The term “habituation” is a non-associative learning event in which there is progressive diminution of behavioral response with repetition of a stimulus. This ‘learning’ is a fundamental or basic process of biological systems and does not require conscious motivation or awareness to occur. Indeed, with habituation a person or an experimental animal is able to distinguish meaningful information from the background, unchanging information.

The term “classical conditioning” also referred to as “Pavlovian conditioning” or “respondent conditioning” is meant a type of learning found in animals, caused by the association (or pairing) of two stimuli.

By “cognitive function” is meant the mental processes by which knowledge (learning) is acquired and stored (memory). These include perception, reasoning, acts of creativity, problem-solving and possibly intuition.

By “cognitive disorders” is meant the set of disorders consisting of significant impairment of cognition or memory that represent a marked deterioration from a previous level of functioning. They can include memory lapses, difficulty concentrating, word mix-ups when speaking or writing, “spaciness,” and clumsiness.

“Candidate agent” or “candidate drug” as used herein describes any molecule, e.g., proteins including biotherapeutics including antibodies and enzymes, small organic molecules including known drugs and drug candidates, polysaccharides, fatty acids, vaccines, nucleic acids, etc. that can be screened for activity as outlined herein. Candidate agents are evaluated in the present invention for a wide variety of reasons, including discovering potential therapeutic agents that affect microtubule polymerization and depolymerization rates, and therefore potential affects on learning and memory; for elucidating toxic effects of agents (e.g., environmental pollutants including industrial chemicals, pesticides, herbicides, etc.), drugs and drug candidates, food additives, cosmetics, etc.; drug discovery; as well as for facilitating basic biomedical research (e.g., research into the fundamental processes of learning and/or memory).

Candidate agents encompass numerous chemical classes. In one embodiment, the candidate agent is an organic molecule, preferably small organic compounds having a molecular weight of more than 100 and less than about 2,500 daltons. Particularly preferred are small organic compounds having a molecular weight of more than 100 and less than about 2,000 daltons, more preferably less than about 1500 daltons, more preferably less than about 1000 daltons, more preferably less than 500 daltons. Candidate agents comprise functional groups necessary for structural interaction with proteins, particularly hydrogen bonding, and typically include at least one of an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression and/or synthesis of randomized oligonucleotides and peptides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

The candidate agents may be proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homophenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations. Peptide inhibitors of enzymes find particular use.

The candidate agents may be naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the systems described herein. Particularly preferred in this embodiment are libraries of bacterial, fungal, viral, and mammalian proteins, with the latter being preferred, and human proteins being especially preferred.

The candidate agents may be antibodies, a class of proteins. The term “antibody” includes full-length as well antibody fragments, as are known in the art, including Fab Fab2, single chain antibodies (Fv for example), chimeric antibodies, humanized and human antibodies, etc., either produced by the modification of whole antibodies or those synthesized de novo using recombinant DNA technologies, and derivatives thereof.

The candidate agents may be nucleic acids. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage, et al., Tetrahedron, 49(10):1925 (1993) and references therein; Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989)), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996), all of which are incorporated by reference)). Other analog nucleic acids include those with positive backbones (Denpcy, et al., Proc. Natl. Acad. Sci. USA, 92:6097 (1995)); non-ionic backbones (U.S. Pat. Nos. 5,386,023; 5,637,684; 5,602,240; 5,216,141; and 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleoside & Nucleotide, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook, and peptide nucleic acids. Nucleic acids containing one or more carbocyclic sugars are also included within the definition of nucleic acids (see Jenkins, et al., Chem. Soc. Rev., (1995) pp. 169-176). Several nucleic acid analogs are described in Rawls, C & E News, Jun. 2, 1997, page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occurring nucleic acids and analogs may be made. The nucleic acids may be single stranded or double stranded, as specified, or contain portions of both double stranded or single stranded sequence, including restriction fragments, viruses, plasmids, chromosomes, etc. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine etc. It should be noted in the context of the invention that nucleosides (ribose plus base) and nucleotides (ribose, base and at least one phosphate) are used interchangeably herein unless otherwise noted.

As described above generally for proteins, nucleic acid candidate agents may be naturally occurring nucleic acids, random and/or synthetic nucleic acids. For example, digests of procaryotic or eucaryotic genomes may be used as is outlined above for proteins. In addition, RNAis are included herein.

“Drug leads” or “drug candidates” are herein defined as chemical entities or biological molecules that are being evaluated as potential therapeutic agents (drugs). “Drug agents” or “agents or “compounds” are used interchangeably herein and describe any composition of matter (e.g., chemical entity or biological factor) that is administered, approved or under testing as potential therapeutic agent or is a known therapeutic agent.

By “effect” is meant an observable change in some objective process or structure within the living system including but not limited to changes in biochemical, metabolic, genetic, cellular, structural, neurological, physiological, electrophysiological, cognitive, social or behavioral metrics or outcomes or in a subjective process such as a symptom as reported by a subject or patient.

By “therapeutic effect” is meant any effect with potential benefit in any disease or condition.

By “time point” and grammatical equivalents thereof is meant a stage in time in a living system, particularly a test system, after which it has been exposed to at least one candidate agent and after which it has been administered at least one isotope-labeled substrate and wherein a sufficient period of time has passed such that the isotope-labeled substrate has been incorporated into at least one tubulin subunit during the formation of a microtubule population. At a given time point, one or more samples are obtained from the test system. The methods of the present invention may utilize more than one “time point.” For example, a first, second, third, fourth, etc. time point may be selected for obtaining additional samples.

By “isotopic incorporation” is meant the degree to which an isotope-labeled substrate has been incorporated into one or more tubulin subunits. According to the methods of the invention, “isotopic incorporation” may be measured in a test or control system. A “test isotopic incorporation” is measured in a sample taken from a test system. A “control isotopic incorporation” is measured in a sample taken from a control system. The measurement of “isotopic incorporation” can be various quantities, including without limitation the content, rate of incorporation and/or pattern or rate of change in content and/or pattern of isotope labeling of the isotope-labeled tubulin subunit(s) that have been incorporate into one or more tubulin subunits. The isotopic incorporation may be measured in population(s) of microtubule molecules and/or in free tubulin subunit(s).

### IV. Methods of the Invention

### A. Overview of the Methods of the Invention

The present invention is directed to methods of determining the dynamics (e.g., the polymerization and depolymerization rates) of microtubules in the cells or tissue of the central nervous system of a living system. First, one or more isotope-labeled substrates (sometimes referred to herein as “precursors”) are administered to the living system for at least a first period of time sufficient to be incorporated into a plurality of subunits (e.g., tubulin dimers and microtubule polymers) of microtubules. The labeled microtubules are obtained from the living system in a variety of ways, for example cell fractionation, and the amount of label is usually quantified. In addition, “unincorporated” labeled substrates can also be quantified; for example using mass spectrometry as outlined herein.

As outlined above, in this manner, the dynamics of microtubules can be determined by measuring and comparing, over specific time intervals, the isotopic content and/or pattern or the rate of change of the isotopic content and/or pattern in the targeted subunit or molecular assemblage (e.g., tubulin dimers and microtubule polymers), for example by using mass spectrometry or other analytical techniques known in the art. The relationship between the isotopic content and/or pattern or the rate of change of the isotopic content and/or pattern in the microtubule to the isotopic content and/or pattern or rate of change in the isotopic content and/or pattern in the unassembled subunits may be particularly informative. (It should be noted, however, that not all systems analysis requires the quantification or evaluation of the “unincorporated” or “free” substrate.) The dynamics of microtubule assembly and disassembly (polymerization and depolymerization) can then be calculated.

Alternatively, radiolabeled substrates are contemplated for use in the present invention wherein the radiolabeled substrates are incorporated into tubulin dimers, which are then incorporated into microtubule polymers. In this manner, the dynamics of microtubules can be determined by measuring radioactivity present in the tubulin dimers and microtubule polymers by using techniques known in the art such as scintillation counting. The dynamics of microtubule polymers are then calculated, using methods known in the art.

In yet another embodiment, both stable and radioactive isotopes are used to label one or more isotope-labeled substrate tubulin dimers.

The targeted microtubule molecule of interest is obtained by biochemical isolation procedures from the cell, tissue, or organism (as discussed more fully, infra), and is identified by mass spectrometry or by other means known in the art. The relative and absolute abundances of the ions within the mass isotopomeric envelope corresponding to each identified microtubule molecule of interest (i.e., the isotopic content and/or pattern of the molecule or the rate of change of the isotopic content and/or pattern of the molecule) are quantified. In one embodiment, the relative and absolute abundances of the ions within the mass isotopomeric envelope corresponding to each identified microtubule molecule of interest are quantified by mass spectrometry. Flux rates through the targeted microtubule pathways are then calculated by use of equations known in the art and discussed, infra. Flux rates through the targeted microtubule pathways are compared in the presence or absence of exposure to one or more candidate agents or combinations of candidate agents, or in response to different levels of exposure to one or more candidate agents, or in response to different levels of exposure to combinations of candidate agents.

In this manner, changes in the targeted microtubule polymerization and/or depolymerization pathway underlying changes in learning and memory and other cognitive functions (or cognitive dysfunctions such as Alzheimer's disease) are measured and quantified and related to disease diagnosis; disease prognosis; therapeutic efficacy of administered candidate agents; or toxic effects of candidate agents.

In another embodiment, the dynamics of microtubules are measured from the polymerization and depolymerization of microtubules in a living organism prior to, and after, exposure to one or more candidate agents, to evaluate toxicity, for example to evaluate whether candidate agents cause or contribute to learning or memory impairment. As will be appreciated by those in the art, a variety of suitable classes of candidate agents may be tested for toxicity, including, but not limited to, industrial or occupational chemicals, cosmetics, food additives, environmental pollutants, drugs and drug candidates, etc.

Alternatively, exposure of a living system to candidate agents and the dynamics of microtubules are compared to the dynamics of microtubules from an unexposed living system of the same species to evaluate toxicity (e.g., the use of multiple living systems to evaluate toxicity such as learning or memory impairment).

Comparisons can also be made at different time points or for different time periods, different doses of candidate agents, different combinations of candidate agents, different “pulse-chase” experiments, or combinations thereof. For example, dose curves can be run, or dose time curves, or matrices thereof.

### B. Administering Isotope-Labeled Precursor(s)

As a first step in the methods of the invention, isotope-labeled precursors are administered.

1. Administering an Isotope-Labeled Precursor Molecule

a. Labeled Precursor Molecules

(1) Isotope Labels

The first step in measuring molecular flux rates involves administering an isotope-labeled precursor molecule to a living system. The isotope-labeled precursor molecule may be a stable isotope or radioisotope. Isotope labels that can be used include, but are not limited to, ²H, ¹³C, ¹⁵N, ¹⁸O, ³H, ¹⁴C, ³⁵S, ³²P, ¹²⁵I, ¹³¹I, or other isotopes of elements present in organic systems.

In one embodiment, the isotope label is ²H.

(2) Precursor Molecules (Isotope-Labeled Substrates)

The precursor molecule may be any molecule having an isotope label that is incorporated into the “monomer” or “subunit” of interest, or it can be the monomer itself. Isotope labels may be used to modify all precursor molecules disclosed herein to form isotope-labeled precursor molecules.

The entire precursor molecule may be incorporated into one or more tubulin dimer subunits. Alternatively, a portion of the precursor molecule may be incorporated into the tubulin dimer subunits.

i. Protein Precursors

A protein precursor molecule may be any protein precursor molecule known in the art. These precursor molecules include, but are not limited to, CO₂, NH₃, glucose, lactate, H₂O, acetate, and fatty acids.

Precursor molecules of proteins may also include one or more amino acids. The precursor may be any amino acid. The precursor molecule may be a singly or multiply deuterated amino acid. For example, the precursor molecule may be one or more of ¹³C-lysine, ¹⁵N-histidine, ¹³C-serine, ¹³C-glycine, ²H-leucine, ¹⁵N-glycine, ¹³C-leucine, ²H₅-histidine, and any deuterated amino acid. Labeled amino acids may be administered, for example, undiluted or diluted with non-labeled amino acids. All isotope-labeled precursors may be purchased commercially, for example, from Cambridge Isotope Labs (Andover, Mass.).

Protein precursor molecules may also include any precursor for post-translationally or pre-translationally modified amino acids. These precursors include but are not limited to precursors of methylation such as glycine, serine or H₂O; precursors of hydroxylation, such as H₂O or O₂; precursors of phosphorylation, such as phosphate, H₂O or O₂; precursors of prenylation, such as fatty acids, acetate, H₂O, ethanol, ketone bodies, glucose, or fructose; precursors of carboxylation, such as CO₂, O₂, H₂O, or glucose; precursors of acetylation, such as acetate, ethanol, glucose, fructose, lactate, alanine, H₂O, CO₂, or O₂; precursors of glycosylation and other post-translational modifications known in the art.

The degree of labeling present in free amino acids may be determined experimentally, or may be assumed based on the number of labeling sites in an amino acid. For example, when using hydrogen isotopes as a label, the labeling present in C—H bonds of free amino acid or, more specifically, in tRNA-amino acids, during exposure to ²H₂O in body water may be identified. The total number of C—H bonds in each non essential amino acid is known—e.g., 4 in alanine, 2 in glycine, etc.

The precursor molecule for proteins may be water (e.g., heavy water). The hydrogen atoms on C—H bonds are the hydrogen atoms on amino acids that are useful for measuring protein synthesis from ²H₂O since the O—H and N—H bonds of proteins are labile in aqueous solution. As such, the exchange of ²H-label from ²H₂O into O—H or N—H bonds occurs without the synthesis of proteins from free amino acids as described above. C—H bonds undergo incorporation from H₂O into free amino acids during specific enzyme-catalyzed intermediary metabolic reactions. The presence of ²H-label in C—H bonds of protein-bound amino acids after ²H₂O administration therefore means that the protein was assembled from amino acids that were in the free form during the period of ²H₂O exposure—e.g., that the protein is newly synthesized. Analytically, the amino acid derivative used must contain all the C—H bonds but must remove all potentially contaminating N—H and O—H bonds.

Hydrogen atoms (e.g., deuterium or tritium) from body water may be incorporated into free amino acids. ²H or ³H from labeled water can enter into free amino acids in the cell through the reactions of intermediary metabolism, but ²H or ³H cannot enter into amino acids that are present in peptide bonds or that are bound to transfer RNA. Free essential amino acids may incorporate a single hydrogen atom from body water into the α-carbon C—H bond, through rapidly reversible transamination reactions. Free non-essential amino acids contain a larger number of metabolically exchangeable C—H bonds, of course, and are therefore expected to exhibit higher isotopic enrichment values per molecule from ²H₂O in newly synthesized proteins.

One of skill in the art will recognize that labeled hydrogen atoms from body water may be incorporated into other amino acids via other biochemical pathways. For example, it is known in the art that hydrogen atoms from water may be incorporated into glutamate via synthesis of the precursor α-ketoglutarate in the citric acid cycle. Glutamate, in turn, is known to be the biochemical precursor for glutamine, proline, and arginine. By way of another example, hydrogen atoms from body water may be incorporated into post-translationally modified amino acids, such as the methyl group in 3-methyl-histidine, the hydroxyl group in hydroxyproline or hydroxylysine, and others. Other amino acid synthesis pathways are known to those of skill in the art.

Oxygen atoms (H₂¹⁸O) may also be incorporated into amino acids through enzyme-catalyzed reactions. For example, oxygen exchange into the carboxylic acid moiety of amino acids may occur during enzyme-catalyzed reactions. Incorporation of labeled oxygen into amino acids is known to one of skill in the art. Oxygen atoms may also be incorporated into amino acids from ¹⁸O₂ through enzyme-catalyzed reactions (including hydroxyproline, hydroxylysine or other post-translationally modified amino acids).

Hydrogen and oxygen labels from labeled water also may be incorporated into amino acids through post-translational modifications. In one embodiment, the post-translational modification already may include labeled hydrogen or oxygen through biosynthetic pathways prior to post-translational modification. In another embodiment, the post-translational modification may incorporate labeled hydrogen, oxygen, carbon, or nitrogen from metabolic derivatives involved in the free exchange-labeled hydrogens from body water, either before or after post-translational modification step (e.g., methylation, hydroxylation, phosphorylation, prenylation, sulfation, carboxylation, acetylation, glycosylation, or other known post-translational modifications).

Protein precursors that are suitable for administration into a subject include, but are not limited to, H₂O, CO₂, NH₃ and HCO₃, in addition to the standard amino acids found in proteins as described, supra.

ii. Water as a Precursor Molecule

Water is a precursor of proteins and many organic metabolites. As such, labeled water may serve as a precursor in the methods taught herein.

H₂O availability is probably never limiting for biosynthetic reactions in a cell (because H₂O represents close to 70% of the content of cells, or >35 Molar concentration), but hydrogen and oxygen atoms from H₂O contribute stoichiometrically to many reactions involved in biosynthetic pathways: e.g.: R—CO—CH2-COOH+NADPH+H₂O→R—CH₂CH2COOH (fatty acid synthesis).

As a consequence, isotope labels provided in the form of H- or O-isotope-labeled water is incorporated into biological molecules as part of synthetic pathways. Hydrogen incorporation can occur in two ways: into labile positions in a molecule (i.e., rapidly exchangeable, not requiring enzyme catalyzed reactions) or into stable positions (i.e., not rapidly exchangeable, requiring enzyme catalysis). Oxygen incorporation occurs in stable positions.

Some of the hydrogen-incorporating steps from cellular water into C—H bonds in biological molecules only occur during well-defined enzyme-catalyzed steps in the biosynthetic reaction sequence, and are not labile (exchangeable with solvent water in the tissue) once present in the mature end-product molecules. For example, the C—H bonds on glucose are not exchangeable in solution. In contrast, each of the following C—H positions exchanges with body water during reversal of specific enzymatic reactions: C-1 and C-6, in the oxaloacetate/succinate sequence in the Krebs' cycle and in the lactate/pyruvate reaction; C-2, in the glucose-6-phosphate/fructose-6-phosphate reaction; C-3 and C-4, in the glyceraldehyde-3-phosphate/dihydroxyacetone-phosphate reaction; C-5, in the 3-phosphoglycerate/glyceraldehyde-3-phosphate and glucose-6-phosphate/fructose-6-phosphate reactions.

Labeled hydrogen or oxygen atoms from water that are covalently incorporated into specific non-labile positions of a molecule thereby reveals the molecule's “biosynthetic history”—i.e., label incorporation signifies that the molecule was synthesized during the period that isotope-labeled water was present in cellular water.

The labile hydrogens (non-covalently associated or present in exchangeable covalent bonds) in these biological molecules do not reveal the molecule's biosynthetic history. Labile hydrogen atoms can be easily removed by incubation with unlabelled water (H₂O) (i.e., by reversal of the same non-enzymatic exchange reactions through which ²H or ³H was incorporated in the first place), however:

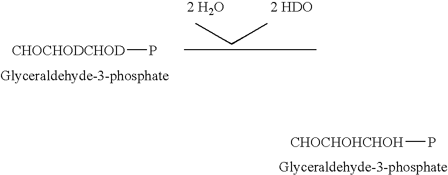

As a consequence, potentially contaminating hydrogen label that does not reflect biosynthetic history, but is incorporated via non-synthetic exchange reactions, can easily be removed in practice by incubation with natural abundance H₂O.

Analytic methods are available for measuring quantitatively the incorporation of labeled hydrogen atoms into biological molecules (e.g., liquid scintillation counting for ³H; mass spectrometry or NMR spectroscopy for ²H and ¹⁸O). For further discussions on the theory of isotope-labeled water incorporation, see, for example, Jungas R L. Biochemistry. 1968 7:3708-17, incorporated herein by reference.

Labeled water may be readily obtained commercially. For example, ²H₂O may be purchased from Cambridge Isotope Labs (Andover, Mass.), and ³H₂O may be purchased, e.g., from New England Nuclear, Inc. In general, ²H₂O is non-radioactive and thus, presents toxicity concerns than radioactive ³H₂O. ²H₂O may be administered, for example, as a percent of total body water, e.g., 1% of total body water consumed (e.g., for 3 litres water consumed per day, 30 microliters ²H₂O is consumed). If ³H₂O is utilized, then a non-toxic amount, which is readily determined by those of skill in the art, is administered.

Relatively high body water enrichments of ²H₂O (e.g., 1-10% of the total body water is labeled) may be achieved relatively inexpensively using the techniques of the invention. This water enrichment is relatively constant and stable as these levels are maintained for weeks or months in humans and in experimental animals without any evidence of toxicity. This finding in a large number of human subjects (>100 people) is contrary to previous concerns about vestibular toxicities at high doses of ²H₂O. One of the Applicants has discovered that as long as rapid changes in body water enrichment are prevented (e.g., by initial administration in small, divided doses), high body water enrichments of ²H₂O can be maintained with no toxicities. For example, the low expense of commercially available ²H₂O allows long-term maintenance of enrichments in the 1-5% range at relatively low expense (e.g., calculations reveal a lower cost for 2 months labeling at 2% ²H₂O enrichment, and thus 7-8% enrichment in the alanine precursor pool, than for 12 hours labeling of ²H-leucine at 10% free leucine enrichment, and thus 7-8% enrichment in leucine precursor pool for that period).

Relatively high and relatively constant body water enrichments for administration of H₂¹⁸O may also be accomplished, since the ¹⁸O isotope is not toxic, and does not present a significant health risk as a result.

iii. Administration of Precursors and Candidate Agents

Isotope-labeled precursors, including water may be administered via continuous isotope-labeled precursor administration, discontinuous isotope-labeled precursor administration, or after single or multiple administration of isotope-labeled precursor administration. In continuous isotope-labeled precursor administration, isotope-labeled precursor is administered to an individual for a period of time sufficient to maintain relatively constant precursor enrichments over time in the individual. For continuous methods, labeled precursor is optimally administered for a period of sufficient duration to achieve a steady state concentration (e.g., 3-8 weeks in humans, 1-2 weeks in rodents).

In discontinuous isotope-labeled precursor administration, an amount of isotope-labeled precursor is measured and then administered, one or more times, and then the exposure to isotope-labeled precursor is discontinued and wash-out of isotope-labeled precursor from body precursor pool is allowed to occur. The time course of delabeling may then be monitored. Precursor is optimally administered for a period of sufficient duration to achieve detectable levels in biological molecules.

Isotope-labeled water may be administered to an individual or tissue in various ways known in the art. For example, isotope-labeled water may be administered orally, parenterally, subcutaneously, intravascularly (e.g., intravenously, intraarterially), or intraperitoneally. Several commercial sources of ²H₂O and H₂¹⁸O are available, including Isotec, Inc. (Miamisburg Ohio, and Cambridge Isotopes, Inc. (Andover, Mass.). The isotopic content of isotope labeled water that is administered can range from about 0.001% to about 20% and depends upon the analytic sensitivity of the instrument used to measure the isotopic content of the biological molecules. In one embodiment, 4% ²H₂O in drinking water is orally administered. In another embodiment, a human is administered 50 mL of ²H₂O orally.

The individual being administered labeled precursor may be a mammal. In one variation, the individual may be an experimental animal including, without limitation, a rodent, primate, hamster, guinea pig, dog, or pig. In variations involving the administering of drugs, drug candidates, drug leads, or combinations thereof, the individual may be a mammal, such as an experimental animal, including an accepted animal model of disease, or a human. In variations involving the administering of food additives, industrial or occupational chemicals, environmental pollutants, or cosmetics, the individual may be any experimental animal such as, without limitation, a rodent, primate, hamster, guinea pig, dog, or pig. Candidate agents may be administered prior to, simultaneously with, or after administration of labeled precursor. Candidate agents are also administered for a sufficient period of time. In addition, different doses of agents may be administered, either to individual animals or to the same animals.

Furthermore, more than one candidate agent may be administered.

### C. Obtaining One or More Targeted Tubulin or Microtubule Polymer Molecules of Interest

In practicing the method of the invention, in one aspect, proteins are obtained from a living system according to methods known in the art.

A plurality of microtubule polymers and/or free tubulin dimer subunits are obtained from the living system using techniques well known in the art of neurobiology. The one or more biological samples may be one or more biological fluids or tissues such as cerebrospinal fluid or nerve tissue. Proteins may be obtained from a specific group of cells, such as neurons, or other growing or non-growing cells. Proteins also may be obtained, and optionally partially purified or isolated, from the biological sample using standard biochemical methods known in the art.

The frequency of biological sampling can vary depending on different factors. Such factors include, but are not limited to, ease and safety of sampling, synthesis and breakdown/removal rates of the proteins, and the half-life of a therapeutic candidate agent administered to a cell, animal, or human.

Proteins may be partially purified and/or isolated from one or more biological samples, depending on the assay requirements. In general, microtubule polymers and/or tubulin dimer subunits may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, fast performance liquid chromatography (FPLC), chemical extraction, thin layer chromatography, gas chromatography, and chromatofocusing. For example, some proteins may be purified using a standard antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the assay and components of the system. In some instances no purification will be necessary.

In another embodiment, the proteins may be hydrolyzed or otherwise degraded to form smaller molecules. Hydrolysis methods include any method known in the art, including, but not limited to, chemical hydrolysis (such as acid hydrolysis) and biochemical hydrolysis (such as peptidase degradation). Hydrolysis or degradation may be conducted either before or after purification and/or isolation of the proteins. The proteins also may be partially purified, or optionally, isolated, by conventional purification methods including HPLC, FPLC, gas chromatography, gel electrophoresis, and/or any other methods of separating chemical and/or biochemical compounds known to those skilled in the art.

### D. Analysis

1. Mass Spectrometry

Isotopic enrichment in proteins can be determined by various methods known in the art such as mass spectrometry, including but not limited to, gas chromatography-mass spectrometry (GC-MS), isotope-ratio mass spectrometry, GC-isotope ratio-combustion-MS, GC-isotope ratio-pyrrolysis-MS, liquid chromatography-MS, electrospray ionization-MS, matrix assisted laser desorption-time of flight-MS, Fourier-transform-ion-cyclotron-resonance-MS, and cycloidal-MS.

Mass spectrometers convert molecules such as proteins into rapidly moving gaseous ions and separate them on the basis of their mass-to-charge ratios. The distributions of isotopes or isotopologues of ions, or ion fragments, may thus be used to measure the isotopic enrichment in a plurality of proteins or phospholipid or cholesterol molecules.

Generally, mass spectrometers include an ionization means and a mass analyzer. A number of different types of mass analyzers are known in the art. These include, but are not limited to, magnetic sector analyzers, electrospray ionization, quadrupoles, ion traps, time of flight mass analyzers, and Fourier transform analyzers.

Mass spectrometers may also include a number of different ionization methods. These include, but are not limited to, gas phase ionization sources such as electron impact, chemical ionization, and field ionization, as well as desorption sources, such as field desorption, fast atom bombardment, matrix assisted laser desorption/ionization, and surface enhanced laser desorption/ionization.

In addition, two or more mass analyzers may be coupled (MS/MS) first to separate precursor ions, then to separate and measure gas phase fragment ions. These instruments generate an initial series of ionic fragments of a protein and then generate secondary fragments of the initial ions.

Different ionization methods are also known in the art. One key advance has been the development of techniques for ionization of large, non-volatile macromolecules including proteins. Techniques of this type have included electrospray ionization (ESI) and matrix assisted laser desorption (MALDI). These have allowed MS to be applied in combination with powerful sample separation introduction techniques, such as liquid chromatography and capillary zone electrophoresis.

In addition, mass spectrometers may be coupled to separation means such as gas chromatography (GC) and high performance liquid chromatography (HPLC). In gas-chromatography mass-spectrometry (GC/MS), capillary columns from a gas chromatograph are coupled directly to the mass spectrometer, optionally using a jet separator. In such an application, the gas chromatography (GC) column separates sample components from the sample gas mixture and the separated components are ionized and chemically analyzed in the mass spectrometer.

In general, in order to determine a baseline mass isotopomer frequency distribution for the protein, such a sample is taken before infusion of an isotopically labeled precursor. Such a measurement is one means of establishing in the cell, tissue or organism, the naturally occurring frequency of mass isotopomers of the protein. When a cell, tissue or organism is part of a population of subjects having similar environmental histories, a population isotopomer frequency distribution may be used for such a background measurement. Additionally, such a baseline isotopomer frequency distribution may be estimated, using known average natural abundances of isotopes. For example, in nature, the natural abundance of ¹³C present in organic carbon is 1.11%. Methods of determining such isotopomer frequency distributions are discussed below. Typically, samples of the protein are taken prior to and following administration of an isotopically

a. Measuring Relative and Absolute Mass Isotopomer Abundances

Measured mass spectral peak heights, or alternatively, the areas under the peaks, may be expressed as ratios toward the parent (zero mass isotope) isotopomer. It is appreciated that any calculation means which provide relative and absolute values for the abundances of isotopomers in a sample may be used in describing such data, for the purposes of the present invention.

2. Calculating Labeled: Unlabeled Proportion of Proteins Such as Microtubule Polymers

The proportion of labeled and unlabeled molecules of interest (e.g., tubulin dimers, microtubule polymers) is then calculated. The practitioner first determines measured excess molar ratios for isolated isotopomer species of a molecule. The practitioner then compares measured internal pattern of excess ratios to the theoretical patterns. Such theoretical patterns can be calculated using the binomial or multinomial distribution relationships as described in U.S. Pat. Nos. 5,338,686, 5,910,403, and 6,010,846, which are hereby incorporated by reference in their entirety. The calculations may include Mass Isotopomer Distribution Analysis (MIDA). Variations of Mass Isotopomer Distribution Analysis (MIDA) combinatorial algorithm are discussed in a number of different sources known to one skilled in the art. The method is further discussed by Hellerstein and Neese (1999), as well as Chinkes, et al. (1996), and Kelleher and Masterson (1992), and U.S. patent application Ser. No. 10/279,399, all of which are hereby incorporated by reference in their entirety.

In addition to the above-cited references, calculation software implementing the method is publicly available from Professor Marc Hellerstein, University of California, Berkeley.

The comparison of excess molar ratios to the theoretical patterns can be carried out using a table generated for a molecule of interest, or graphically, using determined relationships. From these comparisons, a value, such as the value p, is determined, which describes the probability of mass isotopic enrichment of a subunit in a precursor subunit pool. This enrichment is then used to determine a value, such as the value A_{X}*, which describes the enrichment of newly synthesized proteins for each mass isotopomer, to reveal the isotopomer excess ratio which would be expected to be present, if all isotopomers were newly synthesized.

Fractional abundances are then calculated. Fractional abundances of individual isotopes (for elements) or mass isotopomers (for molecules) are the fraction of the total abundance represented by that particular isotope or mass isotopomer. This is distinguished from relative abundance, wherein the most abundant species is given the value 100 and all other species are normalized relative to 100 and expressed as percent relative abundance. For a mass isotopomer M_{X},

$$\begin{matrix}Fractional abundance of {M}_{X} = {A}_{X} \\ = \frac{Abundance {M}_{x}}{{\sum }_{i = 0}^{n} Abundance {M}_{i}} ,\end{matrix}$$

where 0 to n is the range of nominal masses relative to the lowest mass (M₀) mass isotopomer in which abundances occur.

$$\begin{matrix}Δ Fractional abundance ( enrichment or depletion ) = {( {A}_{x} )}_{e} - {( {A}_{x} )}_{b} \\ = {( \frac{Abundance {M}_{x}}{{\sum }_{i = 0}^{n} Abundance {M}_{i}} )}_{e} - \\ {( \frac{Abundance {M}_{x}}{{\sum }_{i = 0}^{n} Abundance {M}_{i}} )}_{b} ,\end{matrix}$$

where subscript e refers to enriched and b refers to baseline or natural abundance.

In order to determine the fraction of polymers that were actually newly synthesized during a period of precursor administration, the measured excess molar ratio (EM_{X}) is compared to the calculated enrichment value, A_{X}*, which describes the enrichment of newly synthesized biopolymers for each mass isotopomer, to reveal the isotopomer excess ratio which would be expected to be present, if all isotopomers were newly synthesized.

3. Calculating Molecular Flux Rates

The method of determining the polymerization and/or depolymerization rate of microtubules includes calculating the proportion of mass isotopically-labeled subunit of a microtubule in the precursor pool, and using this proportion to calculate an expected frequency of a microtubule containing at least one mass isotopically-labeled subunit of a microtubule. This expected frequency is then compared to the actual, experimentally determined isotopomer frequency. From these values, the proportion of microtubule which is formed from added isotopically-labeled precursors during a selected incorporation period can be determined. Thus, the rate of synthesis during such a time period is also determined. In a system at steady-state concentrations, or when any change in concentrations in the system are measurable or otherwise known during said time period, the rate of disassembly is thereby known as well, using calculations known in the art. A precursor-product relationship is then applied. For the continuous labeling method, the isotopic enrichment is compared to asymptotic (e.g., maximal possible) enrichment and kinetic parameters (e.g., synthesis rates) are calculated from precursor-product equations. The fractional synthesis rate (ks) may be determined by applying the continuous labeling, precursor-product formula:

ks=[−ln(1−f)]/t,

where f=fractional synthesis=product enrichment/asymptotic precursor/enrichment

and t=time of label administration of contacting in the system studied.

For the discontinuous labeling method, the rate of decline in isotope enrichment is calculated and the kinetic parameters of subunits are calculated from exponential decay equations. In practicing the method, microtubules are enriched in mass isotopomers, preferably containing multiple mass isotopically labeled subunits of microtubules. These higher mass isotopomers of the microtubule (e.g., proteins containing 3 or 4 mass isotopically labeled tubulin dimers) are formed in negligible amounts in the absence of exogenous precursor (e.g., ²H₂O), due to the relatively low abundance of natural mass isotopically-labeled precursor (e.g., ²H₂O), but are formed in significant amounts during the period of precursor incorporation (e.g., during administration of ²H₂O to the cell, tissue, organ, or organism). The microtubules are taken from the cell, tissue, organ, or organism at the sequential time points and are analyzed by mass spectrometry, to determine the relative frequencies of a high mass isotopomer or to determine the relative frequencies of a high mass isotopomer of a subunit from a microtubule. Since the high mass isotopomer is synthesized almost exclusively before the first time point, its decay between the two time points provides a direct measure of the rate of decay of the subunit. The rate of decay of mass isotopomers that do not contain multiple mass isotopically labeled subunits can also be calculated and used by the methods described herein.

Preferably, the first time point is at least 2-3 hours after administration of precursor (e.g., ²H₂O) has ceased, depending on mode of administration, to ensure that the proportion of mass isotopically labeled subunit (e.g., a labeled tubulin dimer for a microtubule polymer) has decayed substantially from its highest level following precursor administration. In one embodiment, the following time points are typically 1-4 hours after the first time point, but this timing will depend upon the replacement rate of the biopolymer pool.

The rate of decay of the microtubule is determined from the decay curve for the isotope-labeled subunit. In the present case, where the decay curve is defined by several time points, the decay kinetics can be determined by fitting the curve to an exponential decay curve, and from this, determining a decay constant.

Breakdown rate constants (kd) may be calculated based on an exponential or other kinetic decay curve:

kd=[−ln f]/t.

### E. Uses of the Methods of the Present Invention

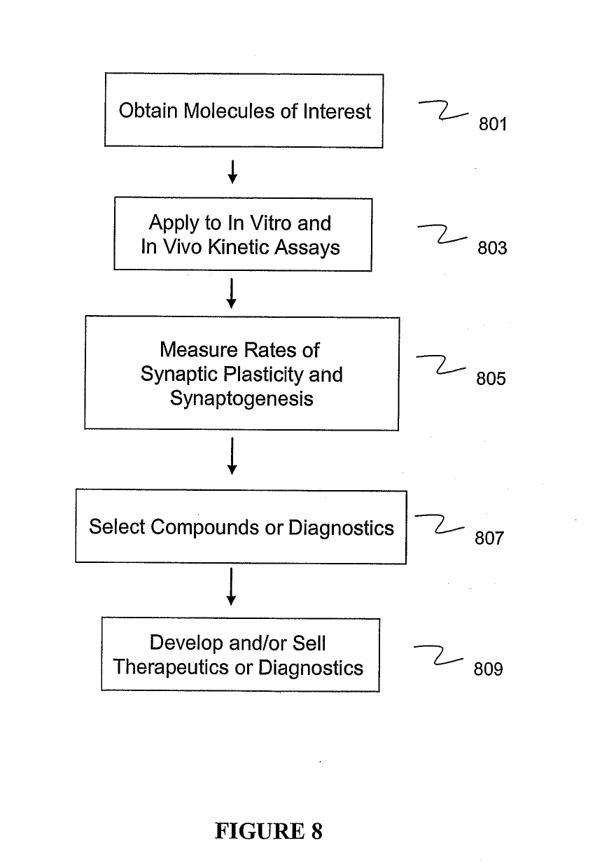

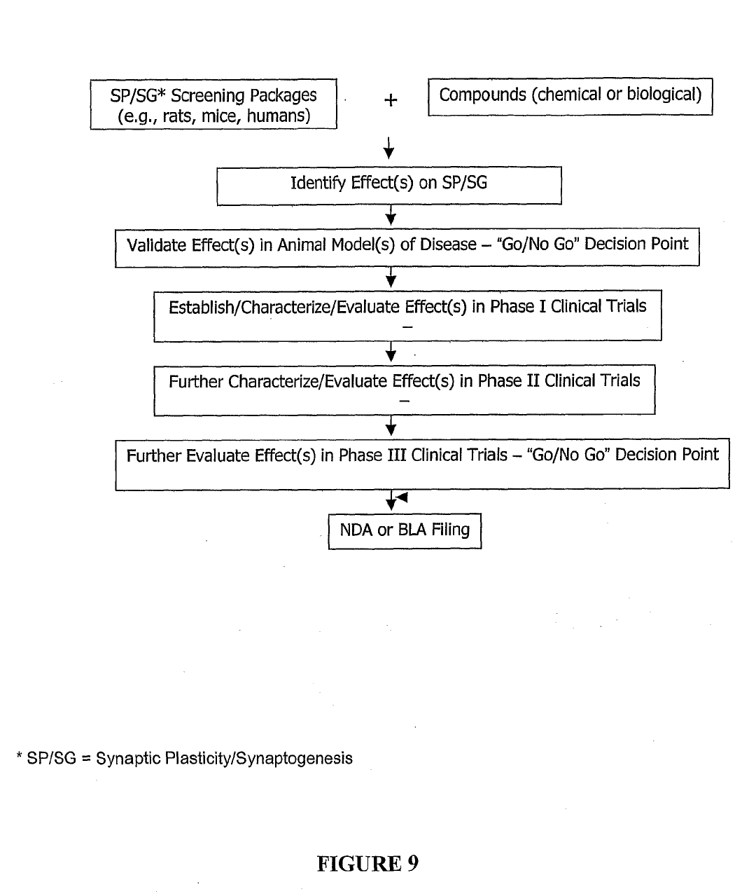

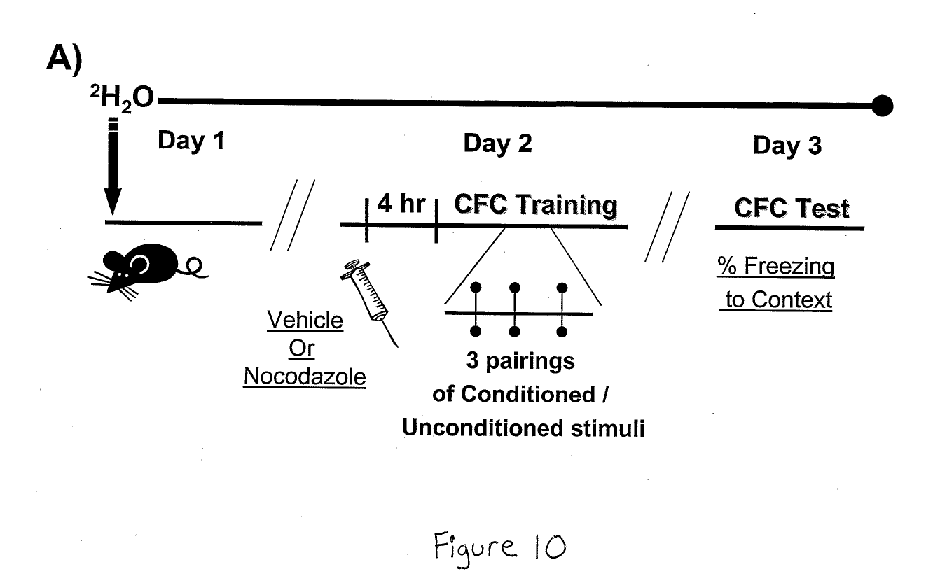

The invention disclosed herein enables the generation of an objective biochemical record of neuronal connectivity (synaptic plasticity) in the living brain which can be monitored by research scientists or clinicians. The invention also allows for the study of the formation of new synapses (synaptogenesis). A particularly valuable use of the invention is the capacity to screen for, select and discard drug candidates that modulate synaptic connectivity, (e.g. synaptic plasticity and/or synaptogenesis) and therefore may improve (or worsen) learning, memory, or other aspects of cognitive function (see FIGS. 8 and 9). This drug screening and filtering approach for modulators of learning and memory is demonstrated to be capable of high-throughput in vivo in animal models.

Standard learning, behavior, and memory animal models including, but not limited to, aversive learning box, swimming or running maze, and Pavlovian conditioning test models are contemplated for use with the methods of the present invention. One of skill in the art could readily use other well known animal models of learning, behavior, and memory with the methods of the present invention in a drug screening project or in basic biomedical research in cognitive function or clinical research in cognitive disorders.

FIG. 8 illustrates the use of the inventions herein in a drug discovery and development process. At step 801, a plurality of candidate agents are obtained (collectively, “molecules of interest”), for example by purchase or in-licensing. At step 803 the molecules of interest are applied to the in vitro and in vivo kinetic assays as described herein. At step 805, synaptic plasticity and/or synaptogenesis are measured as described herein. If it is desirable to increase synaptic plasticity and/or synaptogenesis in a particular phenotypic state, a compound that increases synaptic plasticity and/or synaptogenesis will be considered generally more useful, and conversely a compound that decreases synaptic plasticity and/or synaptogenesis will be considered generally less desirable. In a target discovery process, a particular phenotype that has increased or decreased synaptic plasticity and/or synaptogenesis with respect to another phenotype (e.g., diseased vs. not diseased) may be considered a good therapeutic or diagnostic target or in the pathway of a good therapeutic or diagnostic target. At step 807, molecules of interest or diagnostics are selected and further used and further developed. At step 809, the compounds or diagnostics are sold or distributed. It is recognized of course that one or more of the steps in the process in FIG. 8 will be repeated many times in most cases for optimal results.

FIG. 9 depicts the value of the methods of the present invention in drug discovery and development. Screening initial compounds (i.e., molecules of interest) as described in the preceding paragraph by using the methods of the present invention allows one to select from a set of compounds, a “best in breed” or “best in class” for further development in the costly drug development process. Therefore, the invention enables one of skill to advance one or more candidate agents that are likely to achieve the greatest probability of success in the increasingly more costly and more exacting stages of the drug development process. If a set of compounds yields no promising candidate agent, then the methods of the present invention allows for the decision to terminate further research (the “go/no go” decision point). In the area of drug repurposing or repositioning, the methods of the present invention allow for the screening of compounds that have been extensively tested for, or have already received regulatory approval for marketing, to be screened for activity in stimulating synaptic plasticity and/or synaptogenesis. A compound may stimulate either synaptic plasticity activity or synaptogenesis activity or it may stimulate both. Conversely, a compound may have an inhibitory effect on synaptic plasticity or synaptogenesis or it may inhibit both. The methods of the present invention allow for distinguishing such activities.

Additionally, the methods of the present invention allow for the more in-depth study of candidate agents that have already shown some activity in affecting learning or memory but whose mechanism of action is poorly characterized or not characterized at all. By using the methods of the present invention in such cases, the skilled artisan can choose to go forward with further development (a costly choice should the candidate agent fail later in clinical development) or choose to terminate further development based on the data obtained by screening one or more candidate agents in accordance with the invention herein (again, a “go/no go” decision point). Such information is highly useful in shortening drug development time and saving considerable sums of money.

### F. Isotopically-Perturbed Tubulin or Microtubule Molecules

In another variation, the methods provide for the production of isotopically-perturbed tubulin or microtubule molecules. These isotopically-perturbed molecules comprise information useful in determining their flux within the microtubule assembly/disassembly pathway underlying learning and memory as more fully described supra and infra. Once isolated from a cell and/or a tissue of an organism, one or more isotopically-perturbed molecules are analyzed to extract information as described, supra.

### G. Kits

The invention also provides kits for measuring and comparing molecular flux rates in vivo. The kits may include isotope-labeled precursor molecules, and may additionally include chemical compounds known in the art for separating, purifying, or isolating proteins, and/or chemicals necessary to obtain a tissue sample, automated calculation software for combinatorial analysis, and instructions for use of the kit.

Other kit components, such as tools for administration of water (e.g., measuring cup, needles, syringes, pipettes, IV tubing), may optionally be provided in the kit. Similarly, instruments for obtaining samples from the cell, tissue, or organism (e.g., specimen cups, needles, syringes, and tissue sampling devices) may also be optionally provided.

### H. Information Storage Devices

The invention also provides for information storage devices such as paper reports or data storage devices comprising data collected from the methods of the present invention. An information storage device includes, but is not limited to, written reports on paper or similar tangible medium, written reports on plastic transparency sheets or microfiche, and data stored on optical or magnetic media (e.g., compact discs, digital video discs, optical discs, magnetic discs, and the like), or computers storing the information whether temporarily or permanently. The data may be at least partially contained within a computer and may be in the form of an electronic mail message or attached to an electronic mail message as a separate electronic file. The data within the information storage devices may be “raw” (i.e., collected but unanalyzed), partially analyzed, or completely analyzed. Data analysis may be by way of computer or some other automated device or may be done manually. The information storage device may be used to download the data onto a separate data storage system (e.g., computer, hand-held computer, and the like) for further analysis or for display or both. Alternatively, the data within the information storage device may be printed onto paper, plastic transparency sheets, or other similar tangible medium for further analysis or for display or both.

### I. Examples

The following non-limiting examples further illustrate the invention disclosed herein:

### Example 1

### Cell Culture

The human embryonal carcinoma cell line, NTERA-2 clone DI (NT2), was obtained from the ATCC (Manassas, Va.). Cells were grown and differentiated as described previously (Pleasure, S. J., Page, C., Lee, V. M. Pure, postmitotic, polarized human neurons derived from NTera 2 cells provide a system for expressing exogenous proteins in terminally differentiated neurons. J. Neurosci. 12, 1802-1815 (1992)) with the following modifications: NT2 cells were grown in 75-cm2 tissue culture flasks in complete DMEM (Dulbecco's modified Eagle's medium containing 10% fetal bovine serum, 5% horse serum, 100 U/ml penicillin, and 100 μg/ml streptomycin) in a humidified atmosphere of 10% CO² at 37° C. and differentiated with 10 μM retinoic acid for 3 weeks. Cells were scraped off and replated on a matrigel coated 15-cm² tissue culture dish. After 2 days, neuronal cells were treated with 10 μM fluorodeoxyuridine, 10 μM uridine, and 1 μM cytosine arabinoside for 5 days. The cells were then treated with 10 ng/ml brain-derived neurotrophic factor (BDNF) for a total of 7 weeks. For labeling studies, culture media, as well as the humidified incubator, were adjusted to 5 mol % heavy water (²H₂O) by addition of >99% ²H₂O (Spectra Stable Isotopes, Columbia, Md.) and maintained at this ²H-enrichment for up to 24 h. Where indicated, potassium L-glutamate (Sigma) and adenosine-3′,5′-monophosphorothioate, Rp-isomer (Rp-cAMP, Biolog), or both, were added to cultures at the beginning of the labeling period.

Rat brain hippocampus neuronal cells were obtained from Cambrex Bio Science (Walkersville, Md.). The neurons were grown in Neurobasal medium supplemented with 2 mM Glutamine, 100 U/ml penicillin/streptomycin- and 2% B27 in a humidified atmosphere of 10% CO₂ at 37° C. for 14 days on a poly-D-lysine and laminin coated 10 cm² tissue culture dish.

### Example 2

### Isolation of Tubulin Dimers and Polymers

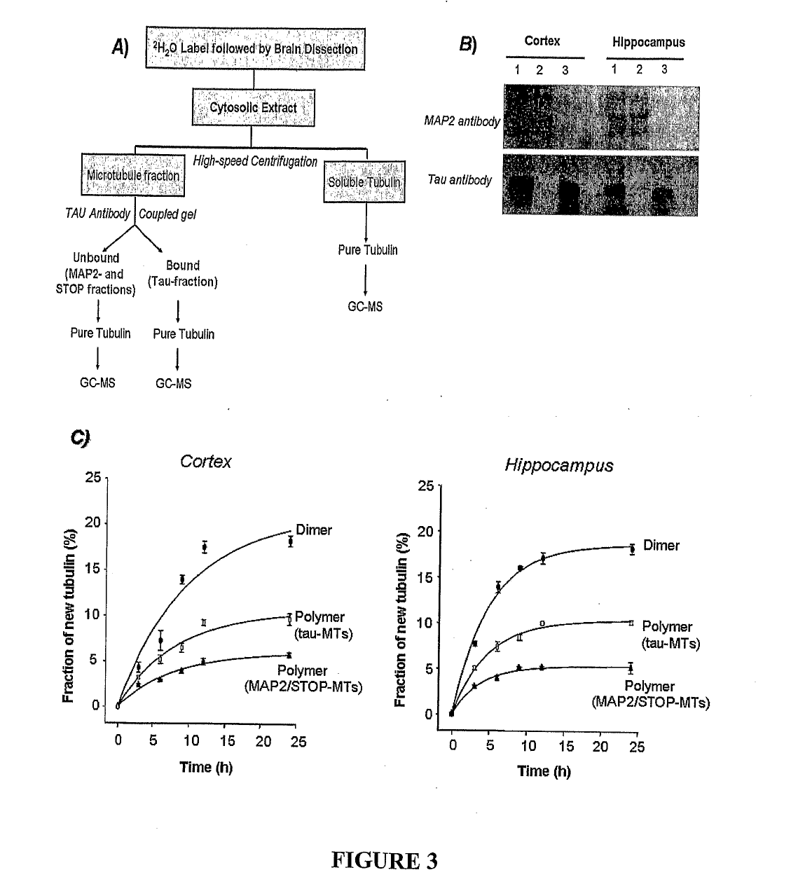

Tubulin was purified using minor modifications of protocols described previously (Fanara, P., Oback, B., Ashman, K., Podtelejnikov, A., Brandt, R. Identification of MINUS, a small polypeptide that functions as a microtubule nucleation suppressor. EMBO J. 18, 565-577 (1999); Fanara, P. et al. In vivo measurement of microtubule dynamics using stable isotope labeling with heavy water. Effect of taxanes. J. Biol. Chem. 279, 49940-49947 (2004). Briefly, cultured neurons were gently scraped off culture plates, washed in phosphate-buffered saline (PBS), and homogenized in microtubule-stabilizing buffer. For purification ex vivo, mice were anesthetized with isoflurane and euthanized by cervical dislocation. Brains were rapidly removed and washed in microtubule-stabilizing buffer (MSB); hippocampi were dissected and gently homogenized in MSB. To separate cytosolic tubulin dimers from microtubule polymers, post-nuclear supernatants were centrifuged at 190,000×g at 20° C. for 35 min. The supernatant or non-microtubule fraction (containing the soluble dimeric tubulin) was separated from the pellet or microtubule fraction (containing polymeric tubulin), quick-frozen and stored at −20° C. Microtubule pellets were further fractionated by sequential immunoaffinity chromatography steps (FIG. 3). In order to isolate tau-associated microtubules, TAU5 antibody was covalently coupled to epoxy-activated Sepharose beads (Amersham Pharmacia Biotech) at a concentration of 0.25 mg/ml. Approximately 0.2 mg of the microtubule pellet was incubated with TAU-5 beads in 0.5 ml MSB for 1 hour at room temperature. Unbound material was removed, the beads were washed three times in 0.5 ml of MSB, and bound material was eluted in 0.5 ml MSB containing 1M NaCl. In some experiments, MAP2-associated microtubules were captured from the TAU5-unbound material by immunoaffinity chromatography on epoxy-activated Sepharose beads coupled to MAP2 antibody (0.5 mg antibody per ml beads) using the same protocol. The relative abundance of tubulin in each preparation (Tubulin dimers and TAU5-bound, MAP2-bound, and unbound microtubule fractions) was quantified by Western blot, and tubulin from these fractions was further purified by ion exchange and size exclusion chromatography, as previously described (Fanara, P. et al. In vivo measurement of microtubule dynamics using stable isotope labeling with heavy water. Effect of taxanes. J. Biol. Chem. 279, 49940-49947 (2004)).

### Example 3

### Isolation of Cold-Stable Microtubules

Cold-stable microtubules were isolated using minor modifications of protocols described previously (Pirollet, F., Derancourt, J., Haiech, J., Job, D., Margolis, R. L. Ca (2+)-calmodulin regulated effectors of microtubule stability in bovine brain. Biochemistry 31, 8849-8855 (1992)). Briefly, cell or tissue crude homogenates were prepared in ice-cold MSB (Fanara, P., Oback, B., Ashman, K., Podtelejnikov, A., Brandt, R. Identification of MINUS, a small polypeptide that functions as a microtubule nucleation suppressor. EMBO J. 18, 565-577 (1999) containing 1.5 mM CaCl2, the proportion of buffer to cell mass or brain tissue was set at a ratio of 1.4:1 (vol/wt). After 2 min. on ice, EGTA was added to a final concentration of 3 mM, and the mixture was homogenized on ice for an additional 1 min. The extract was centrifuged at 150,000×g at 4° C. for 30 min., and the supernatant was collected. Microtubule assembly was initiated by incubating the supernatant at 30° C. After 1 h the extract was chilled at 4° C. for 20 min and centrifuged at 200,000×g for 30 min through a 50% (wt/vol) sucrose cushion in microtubule stabilizing buffer. After suspending the final pellet (cold-stable microtubules) in microtubule destabilizing buffer at 4° C., tubulin was purified as previously described (Fanara, P. et al. In vivo measurement of microtubule dynamics using stable isotope labeling with heavy water. Effect of taxanes. J. Biol. Chem. 279, 49940-49947 (2004)).

### Example 4

### Processing of Tubulin for GC/MS Analysis

Tubulin samples were hydrolyzed by treatment with 6N HCl for 16 hours at 110° C. Protein-derived amino acids were derivatized to pentafluorobenzyl derivatives, and ²H incorporation into alanine was measured by GC/MS as described in detail elsewhere (Fanara, P. et al. In vivo measurement of microtubule dynamics using stable isotope labeling with heavy water. Effect of taxanes. J. Biol. Chem. 279, 49940-49947 (2004)). ²H enrichment was calculated as the percent increase, over natural abundance, in the percentage of alanine derivative present as the (M+1) mass isotopomer.

### Example 5

### Measurement of ²H₂O Enrichment of in Body Water

Body water enrichment of ²H₂O enrichment and culture media was measured as described, supra. Briefly, protons from plasma water were transferred to acetylene by reaction with calcium carbide. Acetylene samples were then analyzed using a Series 3000 cycloidal mass spectrometer (Monitor Instruments, Cheswick, Pa.), which was modified to record ions at m/z 26 and 27 (M₀ and M₁) and calibrated against a standard curve prepared by mixing 99.9% ²H₂O with unlabeled water. Body water ²H enrichments were not affected by drug treatment (data not shown).

### Example 6

### Animal Studies

Male C57BL/6JBomTac mice (Taconic, Germantown, N.Y.), 10 weeks old, were kept in a facility with controlled light-dark cycle, temperature, and humidity. All studies received prior institutional approval. The intracerebroventricular cannulated (ICVC) mice were housed individually. Groups of n=3 ICVC mice were infused through their cannulae connected to a microsyringe by a polyethylene tube. Animals received 6 μl of either 80 μM potassium L-glutamate or 80 μM potassium L-glutamate with 110 nM Rp-cAMP over 6 min. Control animals were injected with sterile water. For ²H₂O labeling, mice then received an intraperitoneal bolus of 30-35 ml/kg ²H₂O (99.9 mol % ²H₂O) containing 0.9% w/v NaCl (Cambridge isotope laboratory, Andover Mass.), resulting in 4-5% body water ²H enrichment, and were maintained on 8% ²H₂O in drinking water (to allow for dilution label by metabolic water) for 24 hours prior to sacrifice. All animals tolerated the treatments well, and there were no differences in body weight among animals in different groups.

### Example 7

### Behavioral Studies

Fear conditioning experiments were done as previously described (Bourtchouladze, R. et al. Different training procedures recruit either one or two critical periods for contextual memory consolidation, each of which requires protein synthesis and PKA. Learn. Mem. 5, 365-374 (1998)). On the training day, C57BL/6JBomTac mice (Taconic), 10 weeks old, received intraperitoneal injections of vehicle (50% cyclodextrin) or nocodazole (0.2 mg/kg) four hours before training and then placed in the fear conditioning chamber (Med Associates) for 2 minutes before the onset of conditioned stimulus, a tone, which lasted for 30 sec at 2800 Hz, 85 dB. The last 2 sec of the conditioned stimulus was paired with the unconditioned stimulus, a 0.5 mA shock. We adopted three pairings of the conditioned with unconditioned stimuli with 1 min intertrial interval. After an additional 30 sec in the chamber, the mice were returned to their home cage. On day later, contextual conditioning was assessed for 3 consecutive minutes in the chamber in which the mice were trained (CFC test). Conditioning was measured by scoring freezing behavior (% of freezing to context), which was defined as complete lack of movement except for respiration, in intervals of 4 sec. Mice were continuously labeled with 8% ²H₂O one day before and during CFC training and test.

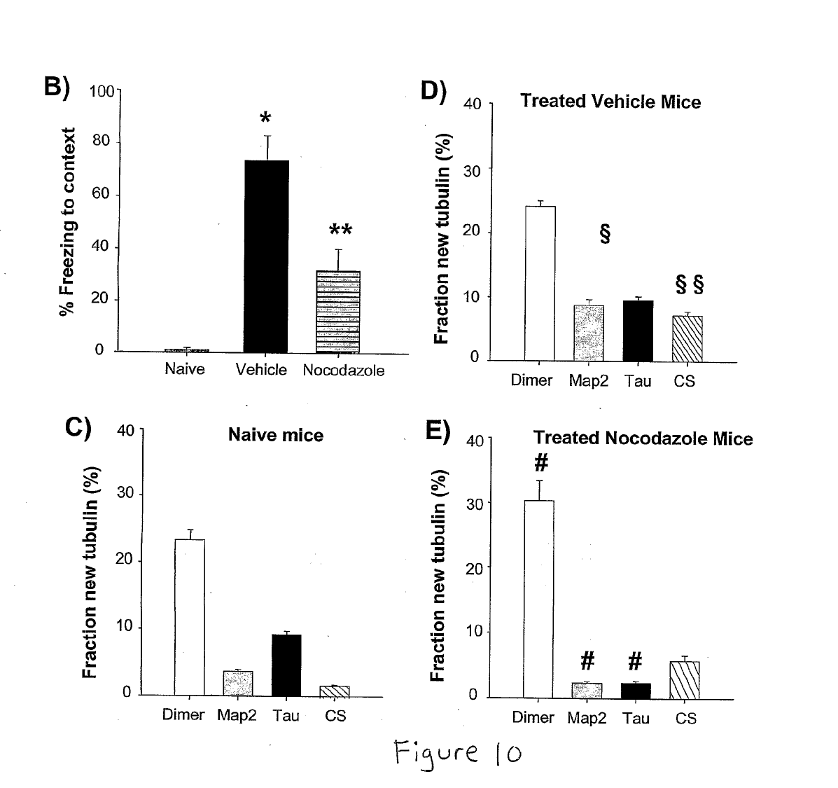

Mice were trained through three pairings of a conditioned stimulus (a tone) and an unconditioned stimulus (an electric shock) and tested for CFC response one day after training (FIG. 10A). To achieve plateau labeling in tubulin dimers, mice were labeled with 8% ²H₂O, starting one day before the CFC training, and the label was continued throughout the experiment (FIG. 10A). Conditioning was measured by recording and scoring freezing behavior (% of freezing to context), which was defined as complete lack of movement except for respiration, in intervals of 4 sec. No naïve animals displayed freezing behavior when exposed to the context (i.e., the chamber in which mice are trained), whereas trained animals exhibit about 80% freezing (FIG. 10B). When labeled with ²H₂O, naïve animals showed a pattern of ²H incorporation into hippocampal free tubulin and microtubule subpopulations similar to that seen before under baseline conditions (FIG. 10C). Interestingly, when exposed to the same context, conditioned animals incorporated more ²H label into MAP2-associated and cold stable microtubules isolated from their hippocampi than did naïve mice (FIG. 10D). The total abundance of these microtubule subsets, as detected by Western blotting, was increased as well, consistent with new assembly of these microtubule subsets during memory formation (data not shown). ²H labeling of tubulin dimers and tau-associated microtubules were unchanged in trained animals. Overall, the rearrangement of different microtubule subpopulations, observed in conditioned animals, closely resembled that seen 24 hours after pharmacological stimulation of LTP using intermediate doses of glutamate, except for the lack of changes in the tau-associated compartment after CFC. Together, these findings reveal that remodeling of MAP2-associated (somatodendritic) and CS (axonal shaft) microtubules are linked to the changes in synaptic efficacy that occur with formation of long-term memory.

To test the role of microtubule/tubulin exchange in hippocampus-dependent memory, we treated animals with nocodazole (0.2 mg/kg), a microtubule-depolymerizing agent, four hours prior to CFC training (FIG. 10A). Interestingly, when exposed to the same context, conditioned animals that were treated with nocodazole showed a significant reduction in freezing behavior compared to the vehicle-treated controls (FIG. 10B). The nocodazole-induced amnesia was due to inhibition of memory formation, rather than prevention of learning, because contextual fear responses measured immediately after training were similar in vehicle- and nocodazole treated animals; moreover, nocodazole treatment caused similar impairment of long-term memory when given 20 minutes after CFC training (data not shown). These behavioral effects were compared to microtubule dynamics in the same animals. Conditioned animals that were treated with nocodazole showed an increase of ²H incorporation into free tubulin dimers (FIG. 10E), as compared to the vehicle-treated controls (FIG. 10D), likely reflecting upregulation of tubulin dimers synthesis (Baas et al., 1991; Jordan et al., 1992). The conditioning-induced increase in label incorporation into MAP2-associated microtubules was reversed by nocodazole. Similarly, label incorporation into tau-associated microtubules was reduced by nocodazole compared to the basal levels found in both naïve and conditioned animals (FIG. 10E). The total abundance of microtubule polymer, as detected by Western blotting, was decreased as well (data not shown). Reduction of label incorporation into CS microtubules was not statistically significant. These findings are in agreement with the reported ability of nocodazole to rapidly disassemble microtubules through association with MT ends in vitro and in cell culture (Baas et al., 1991; Jordan et al., 1992) and suggest that recently assembled, isolated microtubules are disassembled preferentially. Taken together, these data suggest that experimental disruption of microtubule assembly induces amnesia for contextual memory.

### Example 8

### In Vivo Measurement of Microtubule Dynamics in Mouse Brain Using ²H₂O Labeling

Initial studies were performed using the invention disclosed herein to characterize microtubule dynamics within distinct neuronal compartment in vivo in the mammalian brain ²H₂O (8%) was administered in drinking water and animals were sacrificed after 3, 6, 9, 12 and 24 hours of labeling. Brain tissue was removed and dissected into cortex and hippocampus, and cytosolic extracts were fractionated to isolate free tubulin dimers as well as tau-associated (growth cone) and tau-nonassociated (somatodendritic and axonal shaft) microtubules (FIGS. 3, A and B). The tau-nonassociated fraction is thought to consist primarily of somatodendritic and axonal shaft microtubules which are associated with MAP2 and STOP, respectively (Brandt, R. The tau proteins in neuronal growth and development. Front. Biosci. 1, 118-30 (1996); Gonzalez-Billault, C., Engelke, M., Jimenez-Mateos, E. M., Wandosell, F., Caceres, A., Avila, J. Participation of structural microtubule-associated proteins (MAPs) in the development of neuronal polarity. J. Neurosci. Res. 67, 713-719 (2002); Slaughter, T., Black, M. M. STOP (stable-tubule-only-polypeptide) is preferentially associated with the stable domain of axonal microtubules. J. Neurocytol. 32, 399-413 (2003)). To measure rates of new tubulin biosynthesis, tubulin dimers were hydrolyzed, and ²H label incorporation into the nonessential amino acid, Ala, was quantified by GC/MS (Fanara, P. et al. In vivo measurement of microtubule dynamics using stable isotope labeling with heavy water. Effect of taxanes. J. Biol. Chem. 279, 49940-49947 (2004)). Label incorporation into tubulin dimers followed single-exponential kinetics with half-life (t1/2) of about 5-6 hours in the cortex. Label incorporation was about twice as fast (t1/2=3 hours) in the hippocampus (FIG. 3C). After 24 hours, a plateau was reached, at which about 20% of the Ala molecules in tubulin dimers were labeled (FIG. 3C); labeling remained at this level over at least 1 week of continuous labeling with ²H₂O (not shown). Free Ala in brain tissue was >80% equilibrated with ²H₂O (not shown), so the low plateau in new tubulin dimer synthesis (ca. 20% new) most likely represents, the existence of tubulin subpopulations with very slow turnover (t1/2>days). The half-lives of the fast phase of label incorporation into tau-associated (axonal growth cone) and -nonassociated (somatodendritic and axonal shaft) microtubules were similar to the overall turnover rate of tubulin dimers from the same brain region (5-6 hours for cortex and 3 hours for hippocampus). However, plateau labeling of axonal growth cone microtubules reached only about 10%, i.e., half of the labeling observed for free tubulin dimers, and even less (ca. 5%) for other microtubule fractions. The data are consistent with two subpopulations of neuronal microtubules, one that is assembled and disassembled on rapid time scales compared to the fast phase of tubulin dimer synthesis (<a few hours), and one that is almost entirely stable on a time scale of a day. Moreover, the proportion of microtubules in rapid dynamic exchange with dimers appeared to be about twofold higher in axonal growth cones than in other subcellular compartments in the brain. These findings are consistent with the previously measured stability of somatodendritic and axonal shaft microtubules versus the less stable growth cone microtubules in vitro (Baas, P. W., Slaughter, T., Brown, A., Black, M. M. Microtubule dynamics in axons and dendrites. J. Neurosci Res. 30, 134-153 (1991); Tanaka, E., Ho, T., Kirschner, M. W. The role of microtubule dynamics in growth cone motility and axonal growth. J. Cell Biol. 128, 139-155 (1995); Kwei, S. L., Clement, A., Faissner, A., Brandt, R. Differential interactions of MAP2, tau and MAP5 during axogenesis in culture. Neuroreport 9, 1035-1040 (1998); Guillaud, L. et al. STOP proteins are responsible for the high degree of microtubule stabilization observed in neuronal cells. J. Cell Biol. 142, 167-179 (1998); Slaughter, T., Black, M. M. STOP (stable-tubule-only-polypeptide) is preferentially associated with the stable domain of axonal microtubules. J. Neurocytol. 32, 399-413 (2003)).

### Example 9

### Stable Isotope Incorporation into Tubulin During Neurite Elongation and Synaptogenesis

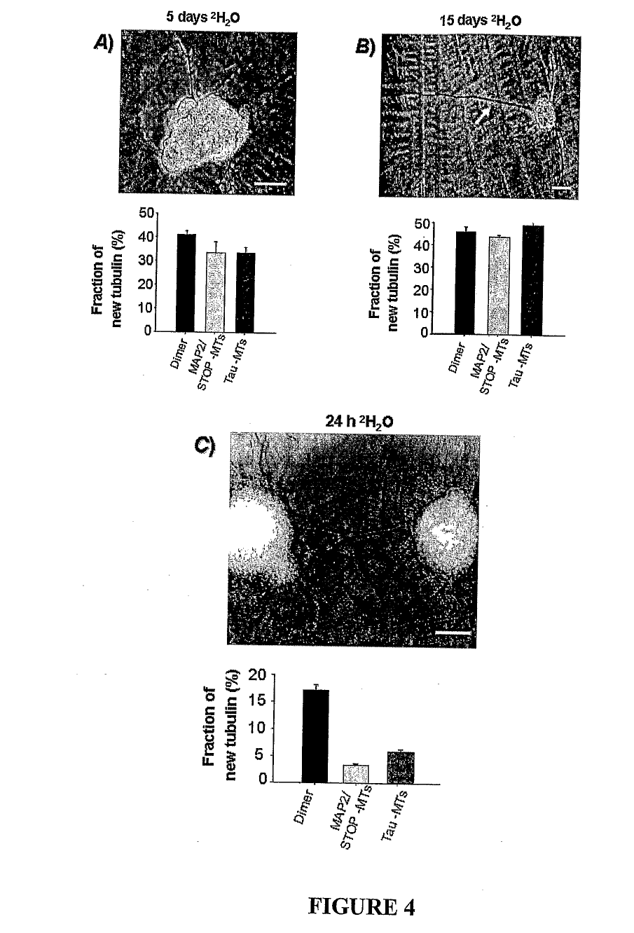

The initial observations of microtubule dynamics in living brain were followed by studies using the invention disclosed herein under more controlled (in vitro) conditions. The microtubule exchange with the free dimer pool was tracked during neuronal differentiation and synaptogenesis in cultured postmitotic NT2-N neuronal cells that were differentiated in vitro using brain-derived neurotophic factor (BDNF). NT2-N neuronal cells were labeled continuously with 5% ²H₂O in culture media for five days after BDNF treatment, at which time the cells can be observed visually to extend multiple neuritic processes (FIG. 4A). At this time, using the ²H₂O labeling technique disclosed herein, 40-50% of Ala residues in tubulin dimers were found to be newly synthesized, indicating a greater contribution of newly synthesized tubulin during in vitro differentiation of neurons than was seen in differentiated neurons from adult rodent brain (cf. FIG. 3C), as expected. Microtubules were almost fully equilibrated with dimers (30-35%) at day 5 (FIG. 4A) indicating that tau-associated and -nonassociated microtubules are highly dynamic during neuronal differentiation in vitro. Indeed, the active microtubule assembly during differentiation may drive up-regulation of tubulin biosynthesis in this model, as the latter is thought to be regulated to match the needs of ongoing microtubule assembly (Cleveland, D. W. Autoregulated control of tubulin synthesis in animal cells. Curr. Opin. Cell Biol. 1, 10-14 (1989); Fanara, P. et al. In vivo measurement of microtubule dynamics using stable isotope labeling with heavy water. Effect of taxanes. J. Biol. Chem. 279, 49940-49947 (2004)).

After 15 days of culture in the presence of BDNF, axonal polarity was clearly established (FIG. 4B). At this time, the fraction of new tubulin dimers had not substantially increased since day 5, suggesting a receding demand for new tubulin synthesis as axonal and somatodendritic polarity is established, and microtubules were fully equilibrated with dimers (FIG. 4B). After 7 weeks of culture with BDNF (FIG. 4C), axons had thickened and formed firm connections to adjacent neural cell clusters (i.e., synaptogenesis had advanced), indicative of terminal neuronal differentiation. ²H₂O labeling was performed over the last 24 hours of these 7-week cultures using the invention disclosed herein. The results demonstrated that microtubule dynamics in these neurons was similar to dynamics seen in hippocampal and cortical neurons in vivo (FIG. 4C, cf. FIG. 3C): about 15-20% of tubulin dimers were labeled, and labeling of somatodendritic and axonal shaft microtubules was less than 5%, indicating that these latter compartments of microtubules were mostly stable; axonal growth cone microtubules were somewhat more dynamic. Thus, use of the invention disclosed herein revealed that the neurotrophic induction of neuronal differentiation in culture is accompanied by an initial burst of newly synthesized tubulin to meet the demands of microtubule assembly during neurite outgrowth; that dynamic remodeling of microtubules accompanies axonal differentiation, culminating in a terminal differentiation state manifesting ongoing synaptogenesis that is characterized by a scaffold of largely stable somatodendritic and axonal shaft microtubules, with growth cones that are somewhat more dynamic. The terminal differentiation state of neurons in cell culture therefore was shown by use of the invention disclosed herein to resemble the microtubule dynamics observed in living adult mammalian brain.

### Example 10

### In Vitro and In Vivo Measurement of Somatodendritic Versus Axonal Microtubule Dynamics

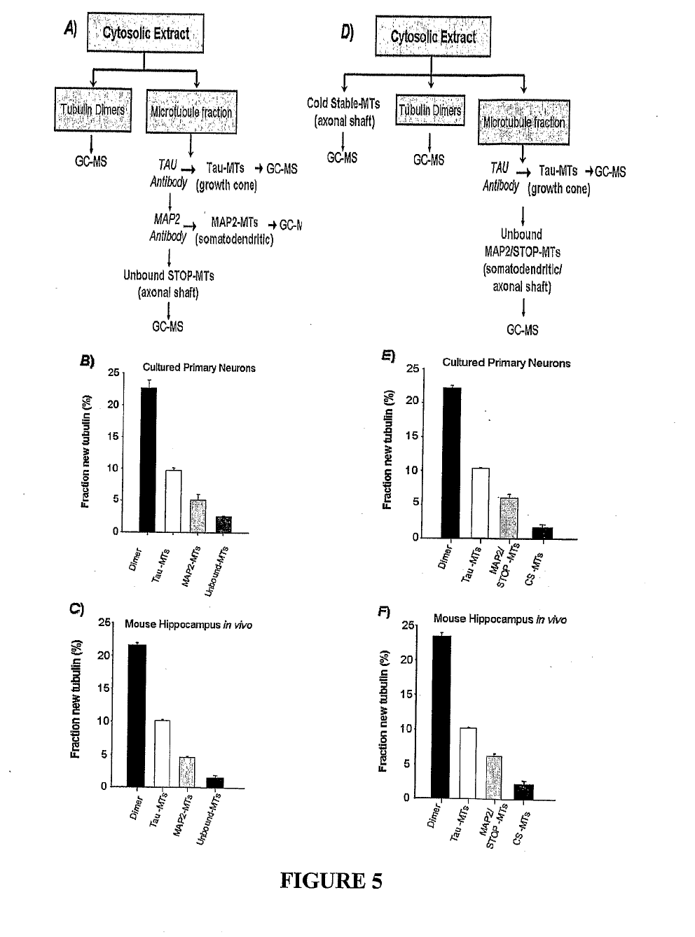

During experimental manipulation of neural connectivity, the dynamic responses of dendritic microtubules to various stimuli are likely to differ from those of axonal shafts or of the cell body. In order to detect such differential responses by use of the invention disclosed herein, we cultured primary neurons from rat brain hippocampi and labeled newly synthesized tubulin by adding 5% ²H₂O in culture media for 24 hours. After separation of tubulin dimers and polymers in cytosolic extracts, we captured axonal growth cone and dendritic microtubules by sequential binding to immunoaffinity columns reactive with tau and MAP2, respectively, leaving only somatic and axonal shaft microtubules in the unbound fraction (FIG. 5A). Tubulin was purified further from each fraction, and label incorporation into Ala was measured by GC/MS (FIG. 5B). About 20-25% of tubulin dimers were newly synthesized in this culture system, a value that is comparable to the values observed in whole hippocampi in vivo or differentiated NT2N cultured cells. Free Ala in culture media was >80% equilibrated with ²H₂O (not shown). Of the microtubule fractions, tau-associated microtubules (axonal growth cone compartment) again were the most highly labeled, and thus the most dynamic, followed by MAP2-associated microtubules (somatodendritic compartment, 4-5% labeled). The tau- and MAP2-nonassociated material was the least dynamic (less than 3% labeled). We obtained the same results with whole mouse hippocampal tissue ex vivo (FIG. 5C).

In order to measure directly the dynamics of axonal shaft microtubules, we exploited their unique ability, compared to other microtubule populations, to resist depolymerization in the cold (Guillaud, L. et al. STOP proteins are responsible for the high degree of microtubule stabilization observed in neuronal cells. J. Cell Biol. 142, 167-179 (1998)), termed “cold-stability”, which has been attributed to their association with STOP proteins (Slaughter, T., Black, M. M. STOP (stable-tubule-only-polypeptide) is preferentially associated with the stable domain of axonal microtubules. J. Neurocytol. 32, 399-413 (2003)). After 24 hours of ²H₂O labeling, cold stable microtubules from cultured primary rat hippocampal neurons (FIG. 5D), or prepared from mouse hippocampi labeled in vivo (FIG. 5E), were reproducibly labeled to a very low extent (<3% new Ala). Of note, the degree of ²H incorporation by cold-stable microtubules (FIGS. 5D and 5E) was similar to that of tau and MAP2-nonassociated microtubules (FIGS. 5B and 5C), suggesting either that axonal shaft microtubules predominate in the latter fraction, or that microtubules of the cell body also exchange poorly with tubulin dimers. We concluded, based on the invention disclosed herein, that association of microtubules with STOP not only prevents cold-induced disassembly in vitro, but also reduces dynamic exchange of axonal shaft microtubules in culture and in vivo, compared to other microtubule subpopulations.

These findings demonstrate that the invention disclosed herein is capable of measuring in vivo the rates of formation and breakdown of the stable microtubules present in the axonal shaft, and thereby to detect the dynamics of the most stable compartment related to neuronal maturation and synaptogenesis.

### Example 11

### Stable Isotope Incorporation Reveals Effects of Glutamate on Microtubule Dynamics During Synaptic Plasticity In Vivo

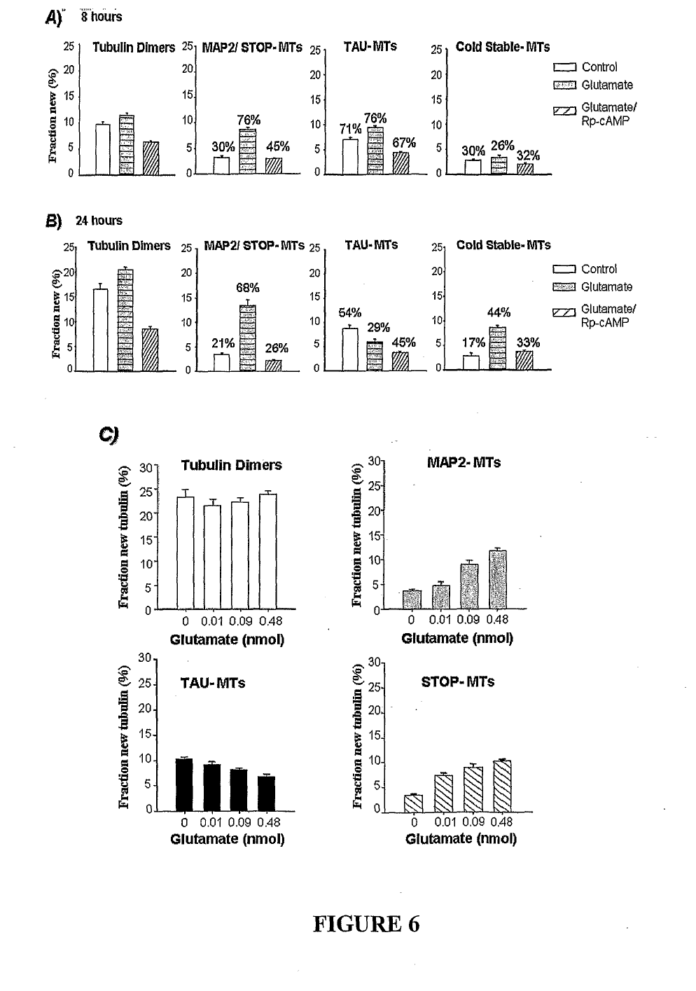

To confirm the ability of the invention disclosed herein to reflect synaptic plasticity in the living brain, we used a neurotransmitter approach based on the work of Wilson (Wilson, M. T., Kisalita, W. S., Keith, C. H. Glutamate-induced changes in the pattern of hippocampal dendrite outgrowth: a role for calcium-dependent pathways and the microtubule cytoskeleton. J. Neurobiol. 43, 159-172 (2000)) demonstrating that glutamate induces dynamic microtubule reorganization and synaptic plasticity in vivo. The model of microtubule dynamic reorganization in vivo during glutamate-induced synaptic plasticity involved infusion ICVC in mice using a previously reported concentration of glutamate (0.48 nmol) (Wilson, M. T., Kisalita, W. S., Keith, C. H. Glutamate-induced changes in the pattern of hippocampal dendrite outgrowth: a role for calcium-dependent pathways and the microtubule cytoskeleton. J. Neurobiol. 43, 159-172 (2000)). Newly synthesized tubulin dimers and exchangeable microtubules were labeled for 8 or 24 h by oral ²H₂O administration, and hippocampal tissue extracts were analyzed for ²H label incorporation into tubulin dimers as well as tau-associated, tau-nonassociated, and cold-stable microtubules (FIG. 6), using the methods of the invention disclosed herein.

As expected, fractional synthesis of tubulin dimers increased from 8 to 24 hours and was somewhat enhanced in glutamate-stimulated animals, likely reflecting increased demands of responding neurons for new microtubule assembly (FIGS. 6A and 6B). Each microtubule fraction exhibited a unique pattern of response to glutamatergic stimulation. Labeling of cold-stable (axonal shaft) microtubules from stimulated animals was indistinguishable from controls at 8 hours but was increased at 24 hours; at the latter time, they were about 40% equilibrated with free tubulin (FIG. 6B). Thus, despite their stability ex vivo, these microtubules can dynamically incorporate substantial amounts of newly synthesized tubulin in vivo, possibly reflecting stimulation of axonal branching (Dent, E. W., Kali, K. Axon branching requires interactions between dynamic microtubules and actin Filaments. J. Neurosci. 21, 9757-9769 (2001)). Microtubules in the tau-nonassociated fraction were highly dynamic (ca. 80% exchanged with free tubulin) both at 8 and 24 hours. Cold-stable microtubules were not dynamic at 8 hours, so the early increase of labeling in the tau-nonassociated fraction (which includes dendritic and axonal shaft microtubules) can be attributed to rapid stimulation of dendritic microtubule assembly. The abundance of MAP2-associated microtubules was increased at 24 hours, as judged by Western blotting (not shown). Conversely, tau-associated microtubules (axonal shaft compartment) were highly exchanged with tubulin dimers 8 hours after stimulation with glutamate, but were actually less dynamic than controls at 24 hours (FIG. 6B). One explanation is that glutamate-stimulated Ca²⁺ flux mobilizes growth cone microtubules, which then re-establish stable connections. It is also possible that between 8 and 24 hours, newly synthesized microtubules may be selectively diverted from the growth cone to supply microtubule assembly in dendrites and axonal branches, leaving unlabeled tubulin populations to be incorporated at the growth cone. Parallel experiments were performed in glutamate-stimulated cultures of primary rat neurons, with identical results (data not shown).

In a separate experiment using the invention disclosed herein, ²H₂O labeling was initiated 24 hours before stimulating animals with varying amounts of glutamate; ²H₂O labeling was continued for another 24 hours until sacrifice (FIG. 6C). In this experiment, tubulin dimers were labeled to plateau by 24 hours (cf. FIG. 3C), and glutamate had at most slight effects on fractional tubulin synthesis. The increased tubulin incorporation into dendritic and axonal shaft microtubules 24 hours post stimulation was shown to be glutamate-dose dependent, as was the decreased label incorporation in tau-associated microtubules at this time (FIG. 6C). Together, these studies using the invention disclosed herein show that glutamate-induced hippocampal synaptic plasticity in the living adult brain triggers a program of sequential microtubule rearrangements in dendrites, axonal shafts, and growth cones through regulated dynamic exchange of microtubules with free tubulin.

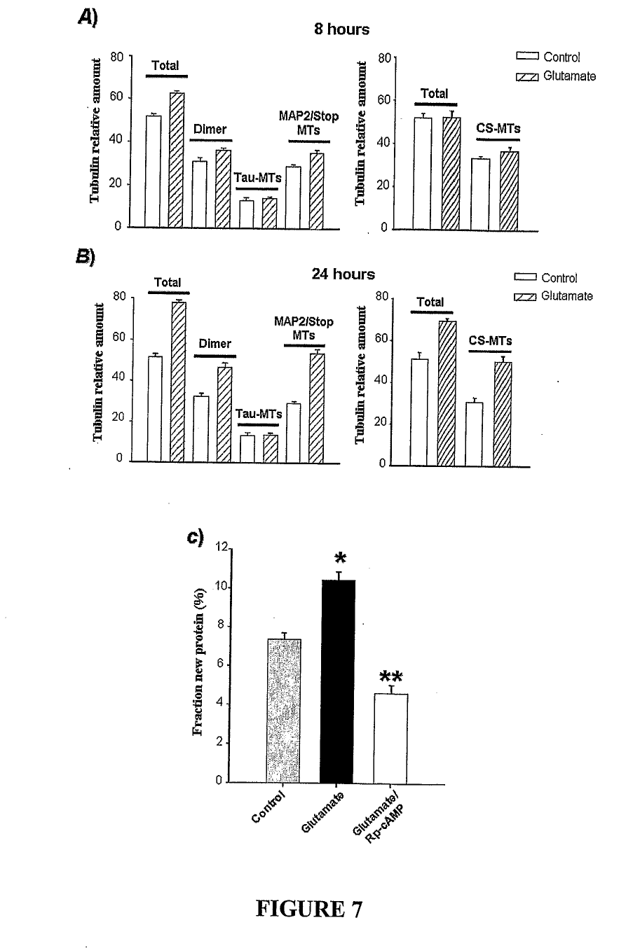

The sensitivity of glutamate-induced synaptic plasticity and hippocampal long-term potentiation (LTP) to inhibitors of protein synthesis has suggested to previous investigators that new protein synthesis is upregulated after glutamatergic stimulation (Frey, U., Morris, R. G. Synaptic tagging and long-term potentiation. Nature 385, 533-566 (1997); Kandel, E. R. The molecular biology of memory storage: a dialogue between genes and synapses. Science 294, 1030-1038 (2001); Wiersma-Meems, R., Van Minnen, J., Syed, N. I. Synapse formation and plasticity: the roles of local protein synthesis. Neuroscientist 11, 228-37 (2005)). This hypothesis had not previously been tested in the living brain, however. To test this hypothesis, we used the methods of the invention disclosed herein. Mice were infused with 6 μl water containing 0.48 nmol glutamate (80 μM in the infusate), or glutamate plus 0.66 pmol Rp-cAMP (110 nM in the infusate). Label incorporation into hippocampal tubulin dimers and into microtubule polymers was then quantified after administration of ²H₂O for 8 hours or 24 hours (FIGS. 7A and 7B). Fractional synthesis of tubulin dimers and microtubules were significantly blocked at 24 hours in the glutamate Rp-cAMP treated versus mock-treated animals (FIG. 7C), suggesting that the glutamate-induced changes in hippocampal microtubule dynamics were dependent on the cAMP/pKA signaling pathway (FIG. 7C). These findings further strengthen the previously reported correlation between hippocampus glutamate NMDA receptor-dependent learning (long-term potentiation) and protein synthesis (Kandel, E. R. The molecular biology of memory storage: a dialogue between genes and synapses. Science 294, 1030-1038 (2001)). By the same criterion, the glutamate-induced program of changes in hippocampal microtubule dynamics shown in FIGS. 6 and 7 also alters hippocampal total protein synthesis in vivo (data not show), further supporting the capacity of the invention disclosed herein to generate a biochemical record of basic neuroanatomic connectivity in the brain and thereby to reveal the dynamics of synaptogenesis in vivo.

We claim:

1. A method for evaluating the effect of a candidate agent on synaptic connectivity in a test system, said method comprising:
a) exposing a test system to at least one candidate agent;
b) administering at least one isotope-labeled substrate to said test system for a period of time sufficient for said isotope-labeled substrate to be incorporated into at least one tubulin subunit during formation of a microtubule population;
c) obtaining from said test system a first sample comprising at least one isotope-labeled tubulin subunit incorporated into a first microtubule population at a first time point and a second sample comprising at least one isotope-labeled free tubulin subunit at said first time point;
d) quantifying a test isotopic incorporation of said isotope-labeled tubulin subunit in said first microtubule population and a test isotopic incorporation of said isotope-labeled free tubulin subunit;
e) providing the quantification of control isotopic incorporation of at least one isotope-labeled tubulin subunit incorporated into a microtubule population from said first time point and of at least one isotope-labeled free tubulin subunit from said first time point;
f) comparing said test and control isotopic incorporations to determine an effect of said agent.

2. The method of claim 1, wherein said quantification comprises measuring the content of isotopic incorporation of said isotope-labeled tubulin subunit incorporated into said microtubule population from said first time point and the content of isotopic incorporation of said isotope-labeled free tubulin subunit from said first time point.

3. The method of claim 1, wherein said comprises measuring the rate of isotopic incorporation of said isotope-labeled tubulin subunit incorporated into said microtubule population from first time point and the rate of isotopic incorporation of said isotope-labeled free tubulin subunit from said first time point.

4. The method of claim 1, 2, or 3, wherein said quantification comprises measuring the pattern of isotopic incorporation of said isotope-labeled tubulin subunit incorporated into said microtubule population from said first time point and the pattern of isotopic incorporation of said isotope-labeled free tubulin subunit from said first time point.

5. The method of claim 1, 2, 3, or 4, wherein said quantification comprises measuring the rate of change in content of isotopic incorporation of said isotope-labeled tubulin subunit incorporated into said microtubule population from said first time point and the rate of change in content of isotopic incorporation of said isotope-labeled free tubulin subunit from said first time point.

6. The method of any one of claims 1-5, wherein said quantification comprises measuring the rate of change in pattern of isotopic incorporation of said isotope-labeled tubulin subunit incorporated into said microtubule population from said first time point and the rate of change in pattern of isotopic incorporation of said isotope-labeled free tubulin subunit from said first time point.

7. The method of claim 1, wherein said comparing step further comprises comparing molecular flux rates in said isotope-labeled tubulin subunit incorporated into said first microtubule population with molecular flux rates in said isotope-labeled free tubulin subunit.

8. The method of claim 1, wherein said comparing step further comprises comparing molecular flux rates in said isotope-labeled tubulin subunit incorporated into said first microtubule population with molecular flux rates in at least one metabolic precursor pool for free tubulin subunits.

9. The method of claim 8, wherein said precursor pool is body water.

10. The method of claim 8, wherein said precursor pool comprises at least one amino acid precursor.

11. The method of claim 1, wherein said isotope is a stable isotope.

12. The method of claim 1, wherein said isotope-labeled substrate is stable isotope-labeled water.

13. The method of claim 1, wherein said isotope-labeled substrate is an amino acid precursor.

14. The method of claim 1, wherein said isotope-labeled substrate is an amino acid.

15. The method of claim 13 or 14, wherein said label is a stable isotope.

16. The method of claim 13 or 14, wherein said label is a radioisotope.

17. The method of claim 15 or 16, wherein said amino acid is selected from the group consisting of ²H-labeled amino acids, ¹³C-labeled amino acids, ¹⁵N-labeled amino acids, ¹⁸O-labeled amino acids, ³H-labeled amino acids, ¹⁴C-labeled amino acids, and ³⁵S-labeled amino acids.

18. The method of claim 1, wherein said test system is exposed to a second candidate agent.

19. The method of claim 1, wherein said test system is exposed to at least one specific dose of said candidate agent.

20. The method of claim 1, wherein said test system is exposed to a second dose of said candidate agent.

21. The method of claim 1, wherein said obtaining step comprises contacting said samples with a microtubule-associated protein binding agent.

22. The method of claim 21, wherein said microtubule-associated protein is selected from the group consisting of tau, Microtubule-Associated Protein2 (MAP2) and Stable Tubule Only Polypeptide (STOP).

23. The method of claim 20, wherein said binding agent is an antibody.

24. The method of claim 1, wherein said effect on synaptic connectivity is a therapeutic effect.

25. The method of claim 24, wherein said therapeutic effect on synaptic connectivity increases cognitive function.

26. The method of claim 24, wherein said therapeutic effect on synaptic connectivity comprises an improvement of at least one clinical sign or symptom of a cognitive disorder.

27. A kit for screening compounds for effects on synaptic connectivity according to the method of claim 1 comprising:
a) at least one isotope-labeled substrate, and
b) instructions for use of said kit.

28. The kit of claim 27 further comprising a tool for administration of said substrate.

29. The kit of claim 27 further comprising an instrument for collecting a sample from a subject.
